(19) United States
(12) Patent Application Publication
(10) Pub. No.: US 2018/0093079 A1
(43) Pub. Date: Apr. 5, 2018

(54) INTERVENTIONAL BALLOON SYSTEM INCLUDING DEFLATION GUIDANCE

(71) Applicant: SurModics, Inc., Eden Prairie, MN (US)

(72) Inventors: Noel Fox, Tourkeel Athenry Co Galway (IE); Joram Slager, St. Louis Park, MN (US); Nathan Allyn Lockwood, Minneapolis, MN (US)

(21) Appl. No.: 15/721,239

(22) Filed: Sep. 29, 2017

Related U.S. Application Data
(60) Provisional application No. 62/402,853, filed on Sep. 30, 2016.

(51) Int. Cl. A61M 25/10

(52) U.S. Cl. CPC A61M 25/104; A61M 25/1027; A61M 2025/1063; A61M 25/1018

(57) ABSTRACT

A balloon system includes a balloon catheter extending from a proximal end portion to a distal end portion. The balloon catheter includes an inflation lumen therein. A balloon assembly is coupled with the distal end portion of the balloon catheter. The balloon assembly includes a balloon having distal and proximal balloon ends, has inflated and folded deflated configurations. One or more deflation guides are coupled with the balloon. The one or more deflation guides are configured to bias the balloon toward the folded deflated configuration.

### PRIORITY CLAIM

This application claims the benefit of priority of U.S. Patent Application Ser. No. 62/402,853, filed on Sep. 30, 2016, which is hereby incorporated by reference herein in its entirety.

### TECHNICAL FIELD

This document pertains generally, but not by way of limitation, to medical devices including balloons.

### BACKGROUND

Medical devices including balloons, such as catheters (e.g., therapeutic, guide, delivery catheters or the like) are used in a variety of medical procedures for instance vascular and body cavity procedures (e.g., gastrointestinal). The balloon is provided in an initial deflated configuration. In some examples the balloon is delivered on its own catheter through a delivery catheter. The balloon is deployed from an end of the delivery catheter and inflated, for instance with saline, a gas or the like.

In some examples, a balloon is inflated within a vessel or body cavity to open the vessel or cavity, for instance as part of a therapeutic procedure where a portion of the anatomy is stenosed and balloon inflation opens the stenosis.

In other examples, a balloon includes one or more coatings such as medicament coatings configured to provide a therapeutic benefit to the patient. Inflation of the balloon in one example provides intimate contact between the balloon and tissue, such as a vessel wall. The drug coating is applied, absorbed or the like from the balloon surface by the tissue according to the intimate contact.

In other examples, a balloon is inflated within a vessel or body cavity to occlude the vessel or cavity, for instance as part of a therapeutic procedure where isolation of a portion of the anatomy is specified. Optionally, the balloon is constructed with compliant materials configured to closely contact the vessel or cavity tissues and provide a reliable seal between the balloon and the tissues.

### OVERVIEW

The present inventors have recognized, among other things, that a problem to be solved can include minimizing the profile of a balloon upon deflation to facilitate retraction of the balloon into a delivery catheter or sheath (e.g., for removal from a patient). When a balloon system (balloon and a balloon catheter) is navigated through a patient to a treatment location, the balloon is provided in an initial deflated configuration (e.g., initial packaged configuration) with the balloon pleated and folded around the shaft of the balloon catheter. Once the balloon is positioned at the treatment site, the balloon is inflated (e.g., with saline, a gas or the like) to an unfolded, expanded configuration (e.g., an inflated configuration) to conduct a procedure including, but not limited to, the application of a medicament, expansion based treatments such as angioplasty or the like. At the conclusion of a procedure the balloon is deflated, retracted into the delivery catheter, and then removed from the patient through the delivery catheter.

During deflation the balloon flattens in an unrestrained fashion, and in at least some examples assumes a flat (e.g., pancake) configuration extending laterally from the balloon catheter. The flat configured deflated balloon is different from the initial packaged (folded) configuration and in some examples has a maximal width or maximal cross-sectional profile greater than an inner diameter of the delivery catheter. Consequently, upon retraction of the deflated balloon into the delivery catheter the balloon scrapes along the surfaces of the delivery catheter (e.g., the edges of the opening). Scraping in some examples abrades the balloon or dislodges coatings present on the balloon, and releases particulate material (e.g., debris). The dislodged debris remains in the vasculature, body cavity or the like after removal of the balloon and in some examples increases the risk of therapeutic complications, such as vascular stenosis or emboli formation.

The present subject matter provides a solution to this problem, such as by providing a balloon system including one or more deflation guides configured to predispose (guide) the deflating balloon away from a configuration with a profile that exceeds the inner diameter of the guiding delivery catheter. For instance, in one example, the one or more deflation guides guide the deflating balloon toward a folded deflated configuration by way of initiating pleats in the balloon (e.g., folds, ridges and grooves or the like). In an example, a folded perimeter of the balloon (when fully deflated) corresponds to the folded profile of the balloon. The folds (preceded by pleats that guide folding) minimize the folded profile of the deflated balloon and facilitate the retraction of the deflated balloon through the distal mouth of the delivery catheter while minimizing scraping and abrading of the balloon. The deflation guides bias the deflating balloon by initiating a pleated guiding configuration, and the pleated guiding configuration guides further deflation of the balloon to the folded deflated configuration. The folded deflated configuration in examples is not identical to the initial packaged configuration (e.g., as made and packaged). The folded deflated configuration includes pleats and folds provided according to post inflation pleating. Optionally, the folds in the folded deflated configuration in other examples are similar to or identical to folds in the initial packaged configuration.

In one example the deflation guides bias the balloon during a portion of deflation of the balloon, for instance at the initiation of deflation. In another example, after the initial bias (e.g., guidance) provided by the deflation guides during initial deflation the deflation guides become passive and thereby apply minimal force to the balloon (e.g., less or none). Accordingly, the one or more deflation guides in an example, provide a tuned force to the balloon that biases the balloon toward pleating (an initial push or pull) while minimizing the application of force to the balloon after a portion of the deflation. Conversely, the one or more deflation guides minimally interact with the inflation of the balloon to the fully deployed configuration (e.g., not at all or with a greatly decreased force relative to initial deflation) thereby ensuring the balloon fully inflates to the desired inflated configuration (e.g., a shape specified for treatment).

The one or more deflation guides include unitary or multi-component deflation guides. In one example, the one or more deflation guides include one or more deflation struts arranged around the balloon. The one or more deflation struts are optionally constructed with an elastomer (e.g., compliant and having a greater elasticity relative to the balloon). Optionally, the deflation struts are coupled at one or more locations along the balloon (e.g., discontinuously, continuously or the like). In another option, the deflation struts are coupled with (e.g., affixed, laid and thereby at least partially movable, or the like) the balloon.

In another example, the one or more deflation guides includes an elastomer shell applied to either of the inner or exterior portion of the balloon (or between layers of the balloon). The elastomer shell is optionally constructed with an elastomer having a greater elasticity and compliance relative to the balloon.

In still another example, the one or more deflation guides include telescoping shafts. For instance, the balloon catheter comprises an exterior shaft coupled (e.g., bonded, engaged with, crimped or the like) with a proximal end portion of the balloon, and an interior shaft coupled with a distal end portion of the balloon. The interior shaft is telescopically received in the exterior shaft, and during deflation the interior shaft is moved distally relative to the exterior shaft (or the exterior shaft is moved proximally) to bias the balloon to pleat and thereby guide folding of the balloon. Optionally, one of the shafts is rotated relative to the other shaft (independently or with longitudinal movement) to bias the balloon to pleat (e.g., helically).

In another option, the one or more deflation guides are provided with another component of the balloon system, such as the delivery catheter. In one example, the delivery catheter includes a braid (e.g., a helical coil, braid or the like) as the one or more deflation guides with a distal portion of the braid coupled near a distal mouth of the delivery catheter. Rotation of the braid relative to the remainder of the delivery catheter (or conversely, rotation of the catheter relative to the braid) causes the braid to expand (e.g., in an unwinding direction) and correspondingly expands the distal mouth of the delivery catheter. The expanded distal mouth readily receives the deflated balloon in a flat configuration or in a folded deflated configuration (e.g., initiated with another example of the one or more deflation guides).

This overview is intended to provide an overview of subject matter of the present patent application. It is not intended to provide an exclusive or exhaustive explanation of the disclosure. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is a schematic view of one example of a balloon system in an initial packaged configuration.

FIG. 1B is a schematic view of balloon system in an inflated configuration.

FIG. 1C is a schematic view of the balloon system of FIG. 1A in a flat deflated configuration prior to retraction into a delivery catheter.

FIG. 1D is a schematic view of the balloon system of FIG. 1C during retraction into the delivery catheter.

FIG. 2 is a plan view of one example of a balloon system.

FIG. 3A is a schematic view of another example of a deployed balloon system including one or more deflation guides in an inflated configuration.

FIG. 3B is a schematic view of the balloon system of FIG. 3A at the initiation of deflation of the balloon with the one or more deflation guides in a pleated guiding configuration.

FIG. 3C is a schematic view of the balloon system of FIG. 3B in a folded deflated configuration.

FIG. 4A is a side view of one example of a portion of a balloon system having one or more deflation guides including one or more deflation struts.

FIG. 4B is a cross sectional view of the balloon system of FIG. 4A.

FIG. 4C is a cross sectional view of another example of a balloon system including one or more deflation struts.

FIG. 4D is a side view of an additional example of a balloon system including one or more deflation struts.

FIG. 4E is a side view of a supplemental example of a balloon system including one or more deflation struts.

FIG. 5A is a longitudinal cross sectional view of another example of a portion of a balloon system having one or more deflation guides including an elastic shell coupled with a balloon.

FIG. 5B is a transverse cross sectional view of the balloon system of FIG. 5A.

FIG. 5C is a cross sectional view of an additional example of a balloon system including an elastic shell between inner and outer layers of the balloon.

FIG. 6A is a partial sectional view showing another example of a portion of a balloon system having one or more deflation guides including telescoping shafts.

FIG. 6B is a partial sectional view of the balloon system of FIG. 6B during initial deflation of the balloon.

FIG. 7A is a partial sectional view showing another example of a portion of a balloon system including a delivery catheter having an expandable distal mouth the one or more deflation guides.

FIG. 7B is a partial sectional view of the balloon system of FIG. 7B with the distal mouth in an expanded configuration while the balloon system is in a deflated configuration.

FIG. 8 is a block diagram showing one example of a method for using a balloon system including one or more deflation guides.

### DETAILED DESCRIPTION

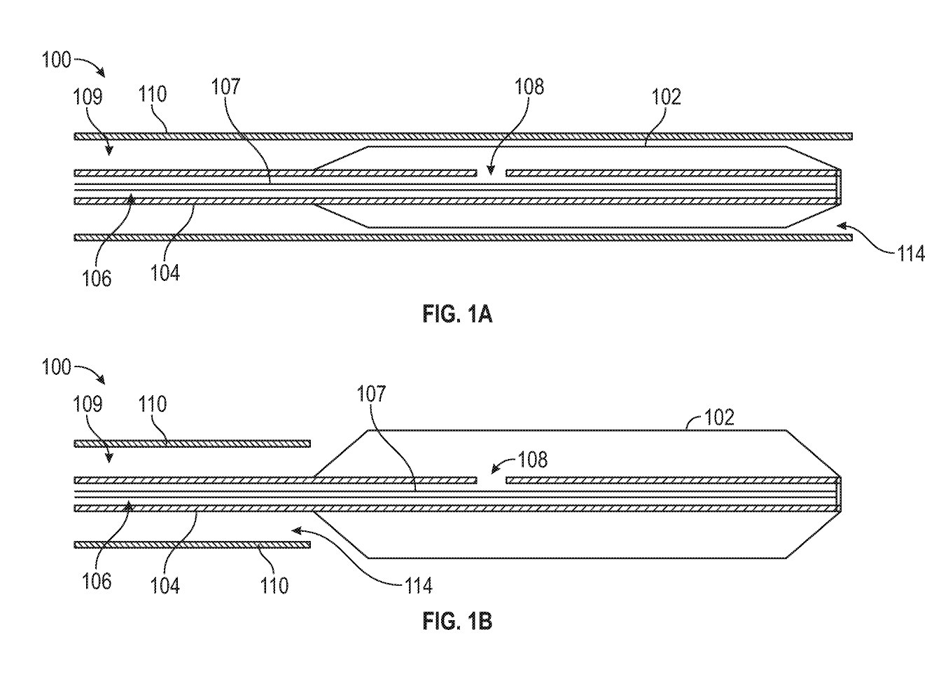

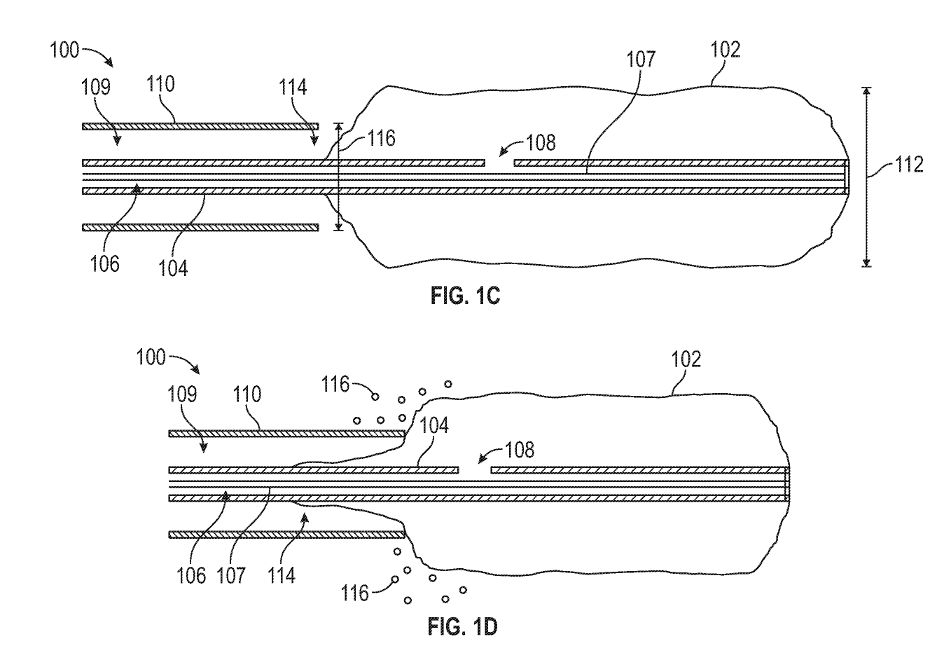

FIGS. 1A-D show one example of a balloon system 100 including a balloon 102 in respective, initial packaged, inflated and flat deflated configurations coupled with a balloon catheter 104.

Referring first to FIG. 1A, the balloon system 100 includes the balloon 102 in the initial packaged configuration (e.g., deflated and compressed or wrapped around the balloon catheter) and received within the delivery lumen 109. As shown the balloon 102 is coupled with the balloon catheter 104 having an inflation lumen 106 therein. The inflation lumen 106 is configured to provide a flow of inflation fluid to the balloon 102 including, but not limited to, one or more of liquid or gas. As further shown in FIG. 1A, in one example the balloon catheter 104 includes an instrument lumen 107 configured to receive one or more instruments including, but not limited to, guidewires, stylets, therapeutic instruments or the like.

As shown in FIG. 1B, the balloon 102 is deployed from the delivery catheter 110. The balloon 102 is shown in close proximity to a distal mouth 114 of the delivery catheter 110. The proximity of the balloon 102 to the distal mouth 114 is exaggerated in this example to compare the profile of the balloon 102 in the inflated configuration shown in FIG. 1B with the mouth profile 116 of the distal mouth 114. After deployment of the balloon 102 the inflation fluid is delivered through the inflation lumen 106 to the inflation port 108 in communication with the interior of the balloon 102. The balloon, constructed in one example with semi-compliant or noncompliant (collectively described as noncompliant) materials is inflated into the inflated configuration shown in FIG. 1B having a profile larger than the corresponding profile of the distal mouth 114 (e.g., the mouth profile 116 shown in FIG. 1C). The mouth profile has a specified shape including, but not limited to, a circular shape, non-circular shape, ovular shape, angled mouth or the like.

Referring now to FIG. 1C, the balloon 102 is shown in a flat deflated configuration, for instance, with the inflation fluid withdrawn from the balloon 102 by way of the inflation port 108 and the inflation lumen 106. As shown in FIG. 1C, the balloon 102 has a flat balloon profile 112 larger than the corresponding mouth profile 116 of the distal mouth 114. In one example, upon deflation of the balloon 102, for instance, from the inflated configuration shown in FIG. 1B the balloon deflates in an unconstrained manner and accordingly assumes the flat deflated configuration shown in FIG. 1C. In this configuration, the material or structure of the balloon membrane extends laterally away from the balloon catheter 104 and accordingly has a larger corresponding profile (the flat balloon profile 112) relative to the mouth profile 116. In one example, the flat balloon profile 112 includes one or more of a width, diameter, radius or the like of the balloon 102 relative to a corresponding dimension of the mouth profile 116 including width, diameter, radius or the like. In another example, the flat balloon profile 112 and the mouth profile 116 are represented by one or more other values, for instance, the cross-sectional area of the mouth profile 116 relative to another dimension of the balloon 102 such as the balloon width, diameter, radius or the like relative to the mouth profile 116 (e.g., in the manner of a ratio).

With the balloon 102 in the flat deflated configuration shown in FIG. 1C, the flat balloon profile 112 is larger than the mouth profile 116. Accordingly, upon retraction of the balloon 102 into the delivery catheter 110 (for extraction of the balloon system 100 from the vasculature, cavity or the like of the subject) as shown in FIG. 1D the balloon 102 engages with the distal mouth 114, for instance, the edges of the delivery catheter 110. In one example, the engagement of the distal mouth 114 with the balloon 102 in the flat deflated configuration 102 causes one or more of shedding, abrasion, dislodgement or the like of a component of the balloon 102. In one example, components of the balloon include, but are not limited to, liners, balloon material, coatings, drug coatings, or the like applied to the balloon 102, for instance for therapeutic purposes. In one example, the abrasion of these components from the balloon 102 frees the components within the vasculature, cavities or the like of the subject as shown with the particulate 116 in FIG. 1D. As previously described herein, in some examples the freeing of these components from the balloon 102 deposits the components in the subject as particulate 116 and accordingly, in other examples, increases the risk of therapeutic complications including emboli or vascular stenosis.

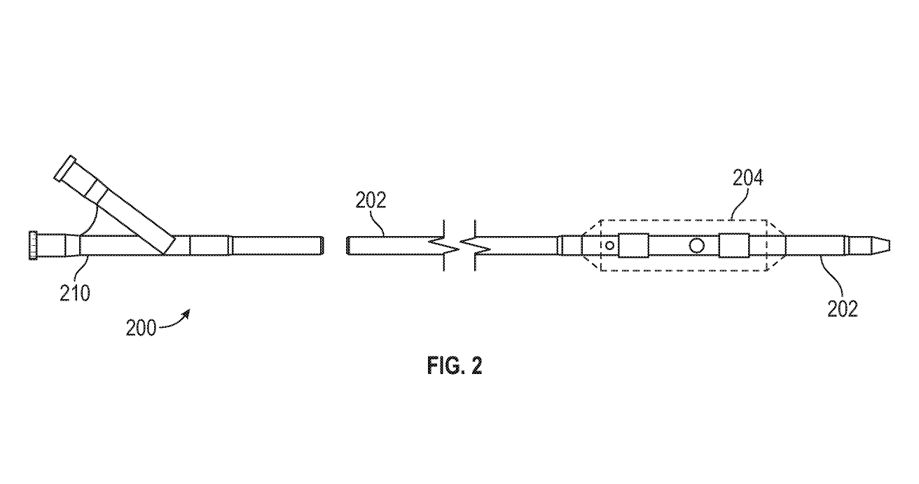

FIG. 2 shows one example of a balloon system 200. For instance, in this example the balloon system 200 includes a balloon catheter 202 and a balloon 204 coupled with the balloon catheter (e.g., as part of an overall balloon assembly 201). Optionally, the balloon system 200 includes additional components including, but not limited to, a delivery catheter, packaging sheath (e.g., for the balloon catheter and balloon) or the like. As shown in FIG. 2, the balloon catheter 202 and the balloon 204 are, in one example, stored within a packaging sheath. As shown in other Figures herein, the balloon catheter 202 is slidably coupled with a delivery catheter. The delivery catheter is, in one example, navigated to a location of interest within a subject, for instance, a particular location in the vasculature, body cavity or the like and the balloon assembly 201 including the balloon catheter 202 and the balloon 204 is deployed from the delivery catheter at the location of interest.

As further shown in FIG. 2, in one example, the balloon catheter 202 is coupled with a system hub 210. In one example, the system hub 210 provides an interface with each of the delivery catheter 206, the balloon catheter 202 and the balloon 204. For instance, the system hub 210 includes an inflation fluid port configured to provide a flow of inflation fluid (as well as evacuation when deflation is desired) of fluid to the balloon 204, for instance, through an inflation lumen such as the inflation lumen 106 shown in FIGS. 1A and 1B. In other examples, the balloon catheter 104 includes additional passages, lumens or the like configured to receive other instruments (e.g., guide wires), fluids such as lytics, medicaments or the like therein for dispensing proximate to the balloon 204. The system hub 210 shown in FIG. 2 in this example includes two ports. In another example, the system hub 210 includes one or more ports (e.g., any number of ports).

Referring again to FIG. 2, the balloon system 200 shown in the figure and further described herein, in one example, includes one or more deflation guides associated with the balloon 204. The deflation guides include one or more features such as struts, shells, telescoping shafts, a deformable expanding mouth (associated with the delivery catheter) or the like configured to facilitate one or more of the retraction or folding of the balloon 204. In another example, the one or more deflation guides facilitate the folding of the balloon 204 into a folded deflated configuration to ensure the corresponding folded profile of the balloon 204 is appropriately sized for non-abrading and non-destructive reception within the delivery catheter 206, for instance, in a distal mouth such as the distal mouth 114 shown in FIGS. 1A-D. Shedding of balloon components including, but not limited to, the balloon material, liners, coating, medicament coatings or the like is accordingly minimized (e.g., eliminated or minimized).

As described herein, in at least some examples, the one or more deflation guides include, but are not limited to, features such as struts, shells, telescoping shafts or the like configured to initiate pleating (e.g., creasing, beginning of folds or the like) of one or more portions of the balloon to thereby facilitate continued deflation (and folding) of the balloon into a folded deflated configuration for reception within the delivery catheter 110 without engagement against the distal mouth 114. Accordingly, large deflated profiles such as the flat deflated configuration 102 shown in FIGS. 1C and 1D are minimized (e.g., eliminated or the likelihood thereof is decreased) to ensure the balloon, such as the balloon 204, is readily retracted into the delivery catheter 206 and one or more of abrasion, dislodgement or the like of the coatings on the balloon, damage to the balloon 204 or the like are substantially minimized.

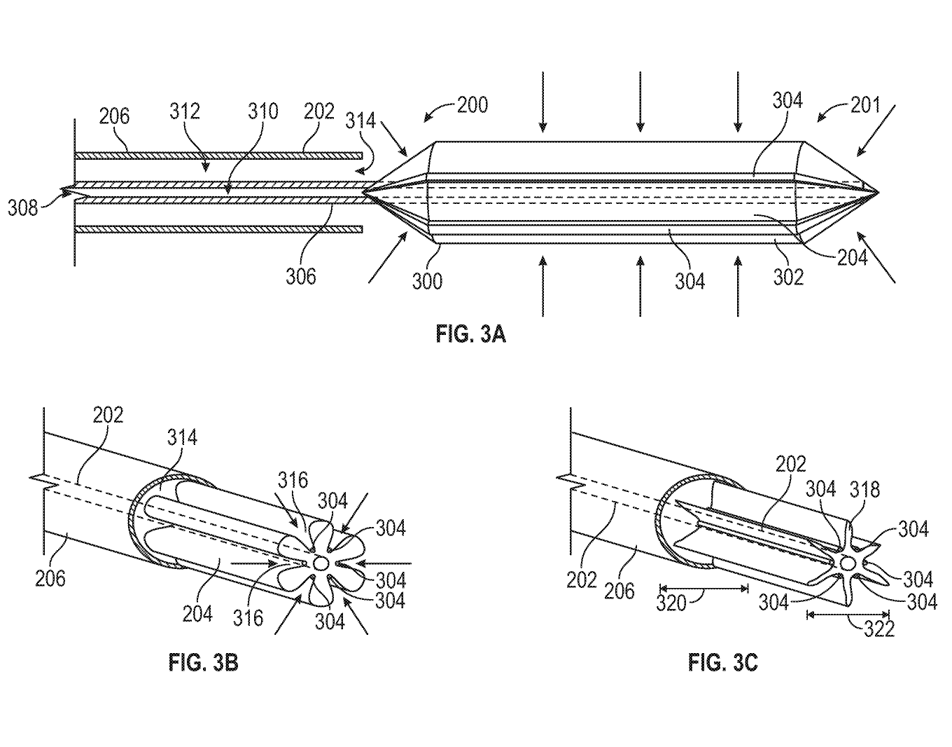

FIGS. 3A, 3B and 3C show the balloon system 200 including the balloon 204 in each of respective an inflated configuration, pleated guiding configuration and folded deflated configuration. Referring first to FIG. 3A, the balloon 204 is deployed relative to the delivery catheter 206 (e.g., distal relative to the distal mouth 314). The balloon 204 is coupled with the balloon catheter 202. For instance, the balloon catheter 202 includes a distal end portion 306 and a proximal end portion 308 (e.g., the proximal end portion 308 is shown in exaggerated close proximity to the distal end portion 306 to facilitate the zoomed in view of the balloon system 200). As further shown in FIG. 3A, a proximal balloon end 300 of the balloon 204 is coupled with the distal end portion 306 of the balloon catheter 202. In one example, the proximal balloon end 300 is coupled with the distal end portion 306 of the balloon catheter 202 with one or more of adhesives, welds, crimps or the like. Similarly, the distal balloon end 302 is coupled with another portion of the distal end portion 306 (distally spaced from the proximal balloon end 300). In a similar manner to the proximal balloon end 300 the distal balloon end 302 is coupled with the balloon catheter 202 with one or more of welds, adhesives, crimps or the like. In one example, the balloon assembly 201 including, for instance, the balloon 204 and the balloon catheter 202 coupled with the balloon 204 are part of an overall balloon system 200 shown in FIG. 3A. Optionally, the delivery catheter 206 is a component of the balloon system 200. In other examples, the balloon system 200 includes the balloon 204, the balloon catheter 202 and the delivery catheter 206 is provided separately.

The balloon 204 is in the inflated configuration, for instance, with inflation fluid provided through the inflation lumen 310 to the balloon 204. The balloon 204 is, in one example, constructed with, but not limited to, noncompliant materials including, but not limited to, polyamides (nylon), polyether-block-amide (PEBAX®, VESTAMID® E), polyurethane or the like. Noncompliant balloons are used in some examples to apply a force to a vessel wall, plaques or the like to dilate the vessel and thereby increase blood flow. In another example, the balloon 204 is constructed with a compliant material, for instance, an elastomer or the like and configured to assume a configuration corresponding to the configuration of a cavity, vessel or the like (e.g., the balloon 204 stretches or deforms). For instance, in one example, the balloon 204 is inflated into the inflated configuration and because of the compliant material of the example balloon material the balloon deforms or stretches into a configuration corresponding to the vasculature (e.g., veins, arteries or the like), cavity or the like of the subject. Compliant balloons are used in other example to occlude a vessel or passage, for instance for a therapeutic procedure downstream from the balloon.

As will be further described herein and as shown in FIG. 3A, each of the balloon systems provided herein includes one or more deflation guides 304 configured to guide deflation of the balloon 204, for instance, into a folded deflated configuration (in one example shown in FIG. 3C). In the example shown in FIG. 3A, the one or more deflation guides 304 include one or more struts, for instance, elastomer deformable struts coupled along the balloon 204. As will be described herein, the deflation guides 304, whether struts, a shell or the like are, in one example, coupled with (applied along) an exterior or interior surface of the balloon 204. In another example, the deflation guides 304 are within the balloon (e.g., coextruded with the balloon material, laminated between layers of balloon material or the like).

The deflation guides 304 are configured to predispose the balloon 204 to deflate into a configuration different from the flat deflated configuration of the balloon 102 shown, for instance, in FIGS. 1C and 1D. Instead, the deflation guides 304 bias (e.g., predispose, preferentially guide, shape or the like) the balloon 204 to assume a folded configuration such as the folded deflated configuration shown in FIG. 3C. In one example, the inflation guides 304 are constructed with a differing material than the balloon 204. For instance, the deflation guides 304 are constructed with an elastomeric material configured to stretch beyond a relaxed configuration (e.g., including one or more of a partially stretched or fully relaxed configuration) during inflation of the balloon 204. The stretching of the one or more deflation guides 304 generates tension within the deflation guides 304 and upon initiation of deflation of the balloon 204 the deflation guides 304 apply a compressive force distributed or localized) around the balloon 204 to guide the balloon 204 to pleat and thereby initiate folding of the balloon 204 into a desired compact configuration, such as the folded deflated configuration shown in FIG. 3C.

Referring now to FIG. 3B, the balloon system 200 previously shown in FIG. 3A is provided in a pleated guiding configuration including a plurality of pleats 316. As shown, the compressive forces (directional arrows) provided around the balloon 204 with the one or more deflation guides 304 initiate pleating or deformation of a portion of the balloon 204 to accordingly predispose the balloon to fold (e.g., into the folded deflated configuration shown in FIG. 3C). The deflation guides 304, such as elastomeric deflation struts, deform the balloon 204 proximate to initiation of deflation of the balloon 204 and pleat the balloon as shown with pleats 316. In another example, the one or more deflation guides 304 such as one or more elastomeric inflation struts (and further including shells, rings, bands or the like as described herein) act on the balloon 204, for instance compressively, to move at least a portion of the balloon 204 toward the balloon catheter 202 and pleat the balloon with the pleats 316. The pleats 316 initiated with the one or more deflation guides 304 guide folding of the balloon 204 into the folded deflated configuration shown in FIG. 3C.

Referring now to FIG. 3C, the balloon 204 is shown in a folded deflated configuration relative to the pleated guiding configuration shown in FIG. 3B. The previously described pleats 316 initiated by the deflation guides 304 guide the folding of the balloon 204 into the folded deflated configuration having one or more fold 318. In another example, he fold 318 are generated in the balloon 204 according to the pleats 316 initiated with the one or more deflation guides 304 shown in FIG. 3B. Continued deflation (e.g., with further withdrawal of the inflation fluid from the balloon 204) transitions the pleats 316 into fold 318 and facilitates the minimizing of the profile of the balloon 204, for instance, to a folded profile 322 shown by way of the dimensional bracket in FIG. 3C. As further shown in FIG. 3C, the distal mouth 314 of the delivery catheter 206 includes a mouth profile 320. As shown, the folded profile 322 of the balloon 204 is smaller than the mouth profile 320 thereby facilitating the retraction of the balloon 204 into the delivery catheter 206 without abrading of the balloon 204 including one or more of dislodging, deformation or damaging of coatings, the balloon material or the like.

Accordingly, with the one or more deflation guides 304 described herein, the balloon system 200 including the deflation guides is configured to guide (e.g., predispose, bias, shape, preferentially dispose) deflation of the balloon 204 into a folded deflated configuration. In one example, the deflation guides 304 bias the balloon 204 at least at the initiation of deflation to assume the pleated guiding configuration. For example, the deflation guides 304 provided at localized positions around the balloon 204 predispose the balloon or bias the balloon to initiate pleating at locations corresponding to the one or more deflation guides 304. After initiating of pleating to form the pleats 316 shown in FIG. 3B, the deflation guides 304 are, in one example, relaxed (fully or partially) and accordingly apply no or minimal further force to the balloon 204. In other examples, the deflation guides 304 maintain some amount of tension (less than at full inflation) even when the balloon 204 is deflated. Continued deflation of the balloon 204 draws the balloon into the folded deflated configuration along the pleats 316 to form the fold 318. The balloon 204 is guided into the folded deflated configuration according to the initial bias provided by the one or more deflation guides 304 to initiate pleating. The pleats 316 provided by the one or more deflation guides 304 correspondingly guide the further folding of the balloon 204 into the folded deflated configuration shown in FIG. 3C. That is to say, in one example, the one or more deflation guides 304 guide the deflation and folding of the balloon 204 by initiating pleating in the balloon 204 and thereafter (through pleating) guide further deflation of the balloon 204 into the folded deflated configuration having a minimized folded profile 322.

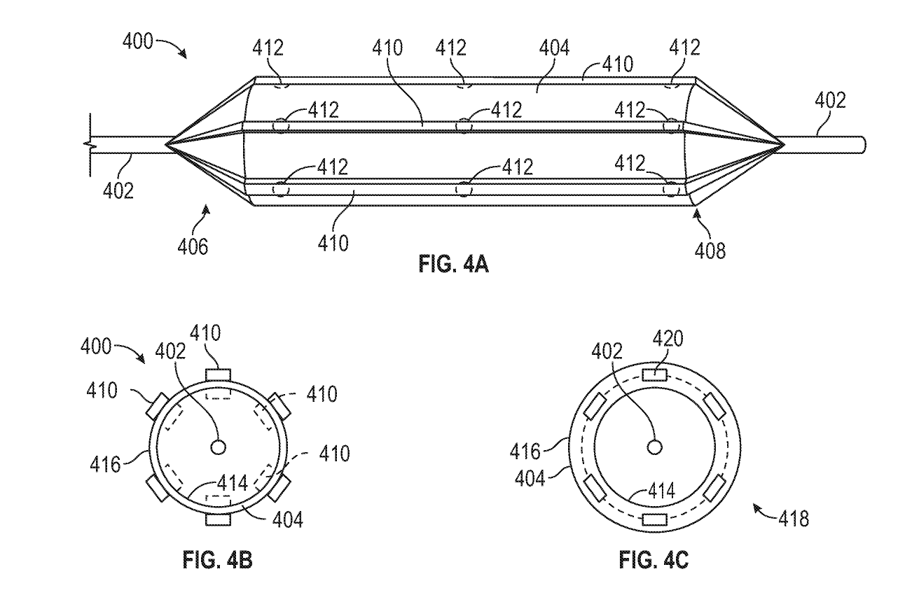

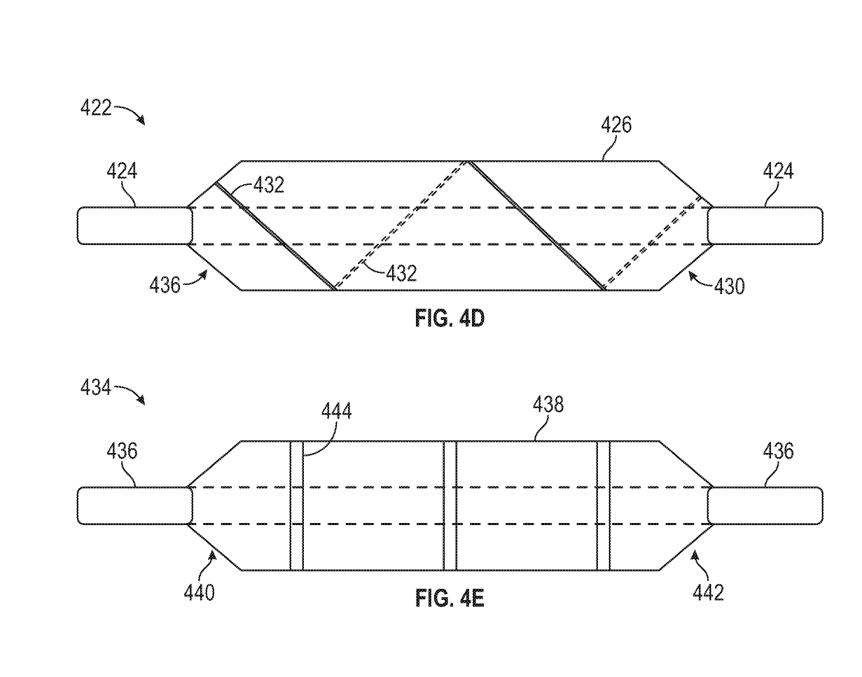

FIGS. 4A, 4B and 4C show two examples of balloon assemblies 400, 418 including one or more deformation guides coupled with the balloon 404 and configured to initiate pleating and guide folding of the balloon into a folded deflated configuration as previously described herein. Referring first to FIG. 4A, a balloon assembly 400 including a balloon 404 coupled with a balloon catheter 402 is shown. The balloon 404 is shown in an inflated configuration to illustrate the one or more deformation guides provided with the balloon 404. The balloon 404 includes a proximal balloon end 406 and a distal balloon end 408. In this example, each of the proximal and distal balloon ends 406, 408 are coupled with the balloon catheter 402, for instance, fixedly attached to the balloon catheter 402 with one or more of adhesives, crimping, welds or the like. As previously described the balloon catheter 402 is configured to provide an inflation fluid to the balloon 404 to inflate the balloon into the inflated configuration shown in FIG. 4A.

As further shown in FIG. 4A, the balloon assembly 400, for instance, the balloon 404 includes a plurality of deflation guides, in this example, deflation struts 410 coupled between the proximal and distal balloon ends 406, 408. In one example, the one or more deflation struts 410 extend continuously between the proximal and distal balloon ends 406, 408. In another example, the one or more deflation struts 410 are coupled between the proximal and distal balloon ends 406, 408, for instance, spaced from one or more of the proximal or distal ends, provided discontinuously between the proximal and distal balloon ends 406, 408 or the like. As previously described, the deflation guides (in this example struts 410) are configured to predispose the balloon 404 to pleat and thereafter fold into a folded deflated configuration such as the folded and deflated configuration shown in FIG. 3C.

In one example, the one or more deflation struts 410 include elastomer deflation struts configured to stretch with inflation of the balloon 404. In one example, the plurality of deflation struts 410 are coupled with a balloon exterior 416 as shown in FIG. 4B. In another example, the one or more deflation struts 410 are provided on the balloon interior 414 (also shown in FIG. 4B in broken lines). In still other examples, the plurality of deflation struts 410 are provided at one or more of the balloon exterior 416 or the balloon interior 414.

As previously described, the deflation struts 410 (e.g., deflation guides as described herein) optionally include one or more elastomer materials. In one example, the deflation struts 410 are constructed with, but not limited to, elastic Nylon, rubber, elastic polyurethane, silicone or the like. With elastomer deflation struts 410 inflation of the balloon 404, for instance from an initial packaged configuration (previously described herein) to the inflated configuration, stretches the deflation struts 410 and accordingly applies a compressive force to the balloon 404. In one example, as the balloon 404 is deflated, (e.g., at the initiation of deflation) the elastomer deflation struts 410 in the stretched configuration pleat the balloon 404 (see FIG. 3B) and the struts relax (fully or partially) as the balloon deforms according to the pleating. The pleats 316 generated in the balloon 404 guide deflation of the balloon 404 to a folded deflated configuration including folds such as the fold 318 shown in FIG. 3C. The folded configuration includes a minimized folded profile 322 (relative to the flat balloon profile shown in FIG. 1C).

In one example, where the deflation struts 410 include an elastomer the deflation struts 410 are slidably coupled along the balloon 404. For instance, as shown in FIG. 4B, the deflation struts 410 are coupled along the balloon, such as the balloon exterior 416, and are free to slidably move longitudinally along the balloon 404 during inflation and deflation of the balloon 404. In another example, one or more anchors 412 are used to fix at least a portion of the deflation struts 410 to a corresponding portion of the balloon 404. For instance, as shown in FIG. 4A, in one example, anchors 412 are provided proximate a midpoint of the balloon 404 and accordingly anchor the deflation struts 410 at that location. The anchors 412 maintain the deflation struts 410 in a distributed arrangement around the balloon 404 and prevent gathering or bunching of the struts. In other examples, the deflation struts 410 are coupled with the balloon 404 with one or more anchors, for instance the anchors 412 shown at various positions along the balloon 404. In the example in FIG. 4A, the anchors 412 are provided proximate the midpoint of the balloon 404 and proximate each of the proximal and distal balloon ends 406, 408. In still other examples, the elastomer deflation struts 410 are continuously coupled or discontinuously coupled along the balloon 404. Accordingly, the deflation struts 410 (e.g., elastomeric or not) are coupled with the balloon 404 movably or fixedly. Optionally, one or more deflation struts 410 are provided along a proximal portion of the balloon, for instance from near the proximal balloon end 406 toward a location between the ends 406, 408. In another option, one or more deflation struts 410 are provided along a distal portion of the balloon from near the distal balloon end 408 toward a location between the ends 406, 408. In still another example, deflations struts 410 are interleaved, with proximal and distal positioned struts provided in a staggered configuration around the balloon 404 (e.g., like interlaced fingers).

Referring now to FIG. 4B, the deflation struts 410 are shown in a distributed arrangement around the balloon 404. In one example, the deflation struts 410 provide a cage or the like around the balloon 404 to accordingly provide compressive forces at one or more of continuous or distributed locations around the balloon 404. In another example, the deflation struts 410 are optionally provided along the balloon interior 414 as also shown in FIG. 4B with broken lines. Although the one or more deflation struts 410 shown in FIGS. 4A and 4B are shown as separate deflation struts, in another example the deflation struts 410 are provided as a composite assembly. For instance, the deflation struts 410 have common junctures or joints. In one example, the common junctures are provided near proximal and distal ends of the balloon 404 proximate to the proximal and distal balloon ends 406, 408. The plurality of deflation struts 410 split and extend separately from the junctures across the balloon 404. The struts 410 optionally then rejoin, for instance, adjacent to the other opposed end of the balloon.

Referring now to FIG. 4C, another example of a balloon assembly 418 is shown including the balloon 404 coupled with a balloon catheter 402 (shown extending through the center of the balloon 404). In this example, the balloon 404 includes the one or more deflation struts 410 within the balloon 404. For instance, the one or more deflation struts 420 are between the balloon exterior 416 and the balloon interior 414. In one example, the plurality of deflation struts 420 are coextruded with the material of the balloon. In still another example, the deflation struts 420 are laminated between layers of the balloon 404 such as the balloon exterior layer corresponding to the balloon exterior 416 and the balloon interior layer corresponding to the balloon interior 414 (the division between the embodiment with two layers is shown with the broken line in FIG. 4C).

FIG. 4D shows another example of a balloon assembly 422 including a balloon 426 coupled with a balloon catheter 424. The balloon 426 is shown in an inflated configuration to illustrate another example of a deflation guide including a deflation strut 432 coupled between the proximal and distal balloon ends 428, 430. As shown in FIG. 4D, the deflation strut 422 extends continuously from the balloon proximal end 428 to the balloon distal end 430. In another example, the deflation strut 432 coupled between the balloon proximal and distal ends 428, 430 includes one or more deflation struts 432 positioned between the proximal and distal ends 428, 430 (e.g., proximate to one or more of the ends, spaced from the ends or the like).

In the example shown in FIG. 4D, the deflation strut 432is provided with a helical shape (e.g., as a helix, coil, spiral or the like) coupled to one or more of the balloon exterior 416, balloon interior 414 (see FIG. 4B) or within the balloon (laminated, coextruded or the like). In another example, the deflation strut 432 is anchored at one or more locations along the balloon 426, for instance, with one or more anchors 412 as shown in FIG. 4A. In another example, a plurality of deflation struts 432 are provided as separated or interconnected coils or the like around the balloon 426. As with the deflation struts 410 and 420 shown in FIGS. 4A and 4C, the deflation strut 432 in one example forms a cage or the like around the balloon 426 and is configured to initiate pleating of the balloon 426. In this example, the pleating follows the shape of the deflation strut 432 and guides folding of the balloon 426 into a folded deflated configuration, for instance, a folded deflated configuration having helical folds.

FIG. 4E shows another example of a balloon assembly 434 including a balloon 438 coupled with a balloon shaft 436. In this example, the one or more deflation struts are coupled with the balloon 438 between the balloon proximal and distal ends 440, 442 and include, but are not limited to, one or more bands, rings or the like provided around the balloon 438. For instance, in one example, the bands or rings are provided on or within the balloon, for instance along the balloon exterior 416, balloon interior 414 (as shown in FIG. 4B) and in other examples within the balloon between the balloon exterior 416 and the balloon interior 414 as described herein previously. The deflation struts 444 including one or more of rings, bands, hoops or the like provided around the balloon 438 predispose the balloon to pleat, for instance into a pleated guiding configuration according to compressive forces applied by the deflation struts 444 to the balloon 438. After initiation of pleating the pleats formed with the deflation struts 444 guide the balloon 438 into a folded deflated configuration to facilitate retraction of the balloon 438, for instance, into a delivery catheter 206 (as shown in FIG. 3A).

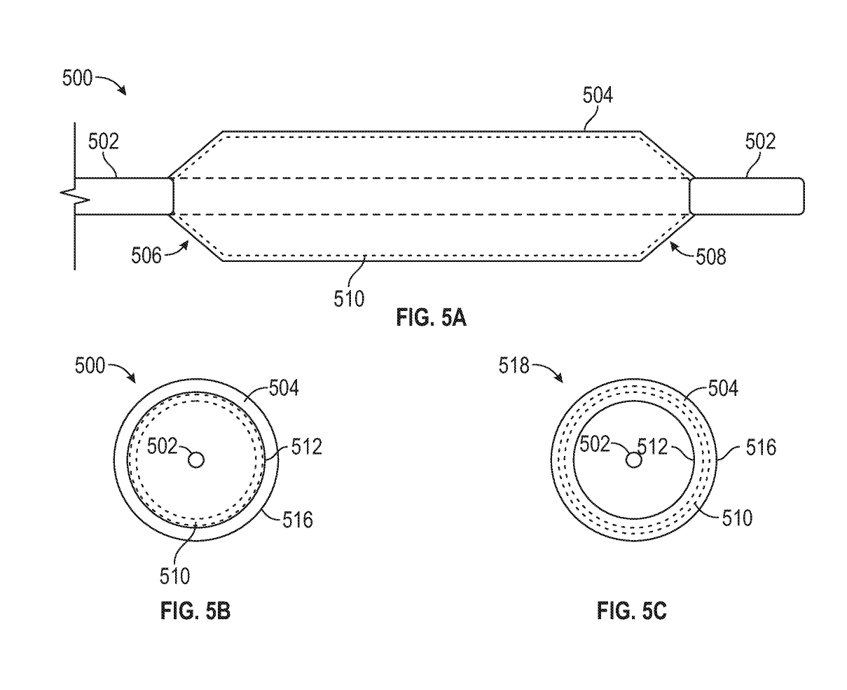

FIG. 5A shows another example of a balloon assembly 500 including a balloon 504 coupled with the balloon catheter 502. As with previous examples, the balloon 504 is shown in an inflated configuration to facilitate viewing of the one or more deflation guides associated with the balloon 504. In this example, the one or more deflation guides include a deflation shell 510 (shown in broken lines in FIG. 5A). Referring again to the figure, the balloon 504 is coupled with the balloon catheter 502, for instance, at a balloon distal end 508 and a balloon proximal end 506. The deflation shell 510 is coupled with the balloon 504 between the balloon distal and proximal ends 508, 506 (continuously, discontinuously, spaced from one of the ends or the like).

In one example, the deflation shell 510 is coupled with one or both of the interior or exterior of the balloon 504. For instance, in one example the deflation shell 510 is provided along the balloon interior 512 as shown in FIG. 5B. The balloon deflation shell 510 is optionally constructed with an elastomer (e.g., one or more of elastic Nylon, silicone, other materials described herein or the like). Coupling of the deflation shell 510 on the balloon interior 512 or within the balloon 504 facilitates the application of one or more coatings, such as medicament coatings along the balloon exterior 516. For instance, the balloon exterior 516 is constructed with a noncompliant or semi-compliant material resistant to stretching that maintains the medicament coating thereon. Instead, the deflation shell 510 stretched along the balloon interior 512 to preserve the coating integrity along the balloon exterior 516.

Referring again to FIG. 5A, the deflation shell 510 extends continuously between the balloon proximal end 506 and the balloon distal end 508. In another example, the deflation shell 510 is discontinuous between the balloon proximal end 506 and the balloon distal end 508 (e.g., includes multiple elements between the ends, is spaced from one or both of the ends or the like).

In another example, the deflation shell 510 like the previously described deflation struts is constructed with an elastomer material, for instance, one or more of the materials previously described herein such as elastic Nylon, silicone, elastic polyurethane, rubber or the like. The deflation shell 510 is configured to stretch with inflation of the balloon 504. For instance, inflation of the balloon 504 (e.g., constructed with one or more of a noncompliant or compliant material) stretches the deflation shell 510. Stretching of the deflation shell 510 and the tension in the shell applies compressive force to the balloon 504 and facilitates the initiation of pleating. In one example, the deflation shell 510 is provided continuously around the perimeter of the balloon 504 and accordingly applies a continuous compressive force inwardly toward the balloon catheter 502 within the balloon 504. In another example, the deflation shell 510 is provided discontinuously, for instance, at one or more radial locations around the perimeter of the balloon 504. In such an example, the deflation shell 510 applies localized inward directed compressive force to the balloon 504, for instance in an arrangement similar to the directional arrows shown in FIG. 3B.

Referring now to FIG. 5B, as previously described, the deflation shell 510 is, in one example, coupled along the balloon interior 512. For instance, the deflation shell 510 is adhered to the balloon interior 512, coextruded with the balloon interior, laminated with the balloon interior or the like. In another example, for instance with a balloon 504 constructed with a pliable material, the deflation shell 510 is provided along the balloon exterior 516. Optionally, the deflation shell 510 is continuously coupled with the balloon 504, for instance, by way of one or more of welds, co-extrusion, adhesion or the like. In another example, the deflation shell 510 is coupled with the balloon 504 with one or more anchors, such as anchors similar to the anchors 412 shown in FIG. 4A. In such an example, the deflation shell 510 is coupled at one or more locations with the balloon 504 and the shell movable between the anchored locations. In one example, the deflation shell 510 is able to stretch and relax relative to the balloon 504 with corresponding forces applied to the balloon 504, for instance, at each of the anchors 412, between the anchors or the like. In still other examples, the deflation shell 510 is coupled with one or more seams including, but not limited to, longitudinally extending seams, radial seams or the like.

FIG. 5C shows another example of a balloon assembly 518 including the balloon 504 positioned around the balloon catheter 502 (according to the cross-section). In the example shown in FIG. 5C, the deflation shell 510 is provided within the balloon 504, for instance, between the balloon interior 512 and the balloon exterior 516. In an example, the deflation shell 510 is coextruded with each of the portions of the balloon 504, for instance, the portions corresponding to the balloon exterior 516 and the balloon interior 512. As shown in FIG. 5C, the deflation shell 510 is a continuous layer provided between the balloon interior and exterior 512, 516. In another example, the deflation shell 510 is a discontinuous shell, for instance, having one or more spaces between each of the shell elements in a manner similar to the deflation struts 420 shown in FIG. 4C. Optionally, the deflation shell 510 is interposed (positioned) between the balloon interior 512 and the balloon exterior 516 and is movable relative to the interior and exterior during inflation and deflation (e.g., the shell is not bonded with one or more of the interior or the exterior).

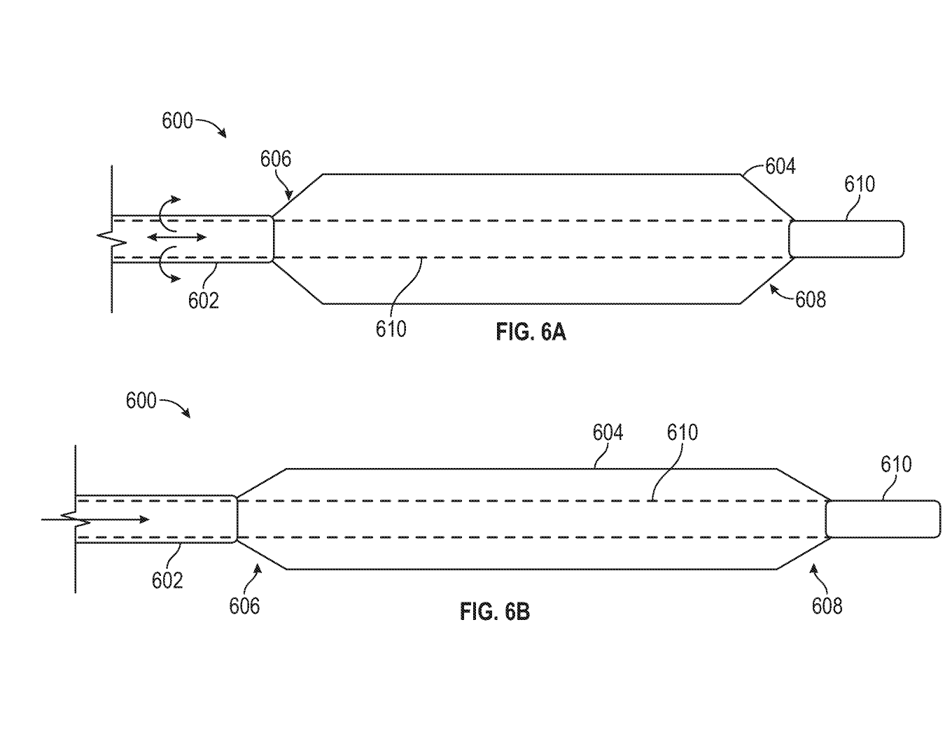

FIGS. 6A and 6B show another example of a balloon assembly 600. In the example shown, the balloon assembly 600 includes the balloon 604 coupled with a balloon catheter 602 and a telescoping shaft 610 (another example of a deflation guide). In one example, the telescoping shaft 610 is an interior shaft slideable relative to the balloon catheter 602 (e.g., an exterior shaft). The balloon catheter 602 is, in one example, coupled with the balloon proximal end 606 and the telescoping shaft 610 is coupled with the balloon distal end 608. The balloon proximal and distal ends 606, 608 are coupled with the respective portions of the balloon catheter 602 and the telescoping shaft with one or more of adhesives, crimps, welds, bonds or the like. Accordingly, with movement of the telescoping shaft 610 relative to the balloon catheter 602 (or conversely the movement of the balloon catheter relative to the telescoping shaft 610) the space between the balloon distal end 608 and the balloon proximal end 606 is changed.

Referring first to FIG. 6A, the balloon 604 is shown in an inflated configuration with the balloon proximal end 606 spaced from the balloon distal end 608 according to a first orientation of the telescoping shaft 610 relative to the balloon catheter 602 (e.g., one or more of rotational or longitudinal positions). In the example shown in FIG. 6A, this particular orientation of the balloon proximal and distal ends 606, 608 (e.g., by positioning of the telescoping shaft 610) facilitates the inflation of the balloon 604 to the inflated configuration (e.g., to a specified shape and size for operation of the balloon).

When deflation is desired, the telescoping shaft 610 is moved, for instance, into a second orientation, for instance shown in FIG. 6B. The telescoping shaft 610 is moved distally relative to the balloon catheter 602 to accordingly position the balloon distal end 608 away from the balloon proximal end 606 relative to the first orientation shown in FIG. 6A. The second orientation shown in FIG. 6B accordingly positions the balloon distal end 608 away from the balloon proximal end 606 and spreads the balloon 604 longitudinally therebetween. The increased spacing between the proximal and distal ends 606, 608 facilitates the pleating of the balloon 604. For instance, as inflation fluid is withdrawn from the balloon 604 and the telescoping shaft 610 is moved distally relative to the balloon catheter 602 the lengthened balloon 604 pleats according to the movement of the telescoping shaft 610. The movement of the telescoping shaft 610 positions the balloon assembly in a pleat initiating configuration that biases the balloon 604 to pleat, for instance, at one or more locations provided around the balloon 604.

In one example, where the balloon distal end 608 is moved distally relative to the balloon proximal end 606, the pleats generated in the balloon 604 are aligned, for instance, with the telescoping shaft 610 and the balloon catheter 602 (e.g., parallel in one example). In another example, the balloon assembly 600 is moved from the inflated configuration shown in FIG. 6A to a pleat initiating configuration by rotation of the telescoping shaft 610 relative to the balloon catheter 602. The telescoping shaft 610 is rotated into a second orientation (for instance, clockwise or counterclockwise) from an initial position to twist the balloon 604 coupled with the balloon catheter 602. The rotation of the telescoping shaft 610 and corresponding twisting of the balloon 604initiates pleating in the balloon 604. In this example, rotation of the telescoping shaft 610 (whether alone or together with longitudinal movement of the telescoping shaft) initiates helical pleating (e.g., helical, spiral, pleating or the like) in the balloon 604. Continued deflation of the balloon 604 folds the balloon 604 according to guidance provided by the pleating initiated with movement of the telescoping shaft 610.

As described herein, the telescoping shaft 610 is another example of a deflation guide associated with the balloon 604 of the balloon assembly 600. The telescoping shaft 610 is optionally used in combination with one or more of the other examples of deflation guides provided herein including, but not limited to, deflation struts, deflation shells or the like.

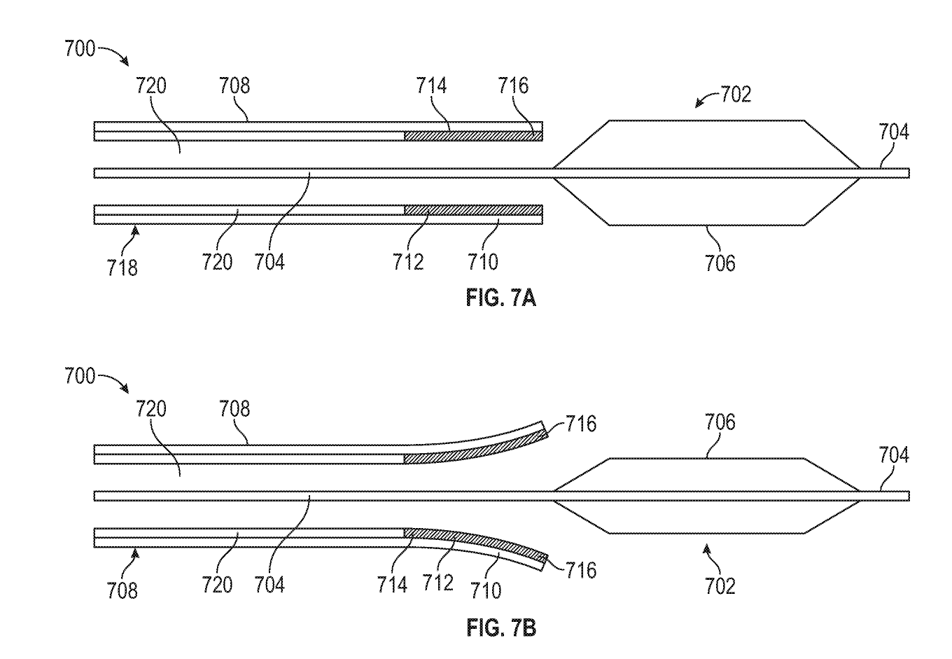

FIGS. 7A and 7B show another example of a balloon system 700 including another example of a deflation guide. In this example, a deflation guide is associated with a delivery catheter 708. Referring first to FIG. 7A, the balloon assembly 702, including for instance, the balloon catheter 704 and the balloon 706, is provided in an inflated configuration and deployed relative to delivery catheter 708. As further shown, FIG. 706 is deployed through a distal mouth such as a deformable distal mouth 710 of the delivery catheter 708. As shown, the deformable distal mouth 710 is in a resting (unexpanded) configuration.

As further shown in FIG. 7A, the delivery catheter 708 includes an expansion mechanism 712 (e.g., another example of a deflation guide) coupled with an interior portion of the delivery catheter 708. In the example shown, a distal mechanism portion 716 of the expansion mechanism 712 is coupled with the deformable distal mouth 710 and operation of the expansion mechanism expands the deformable distal mouth 710. The expansion mechanism 712 includes, but is not limited to, a braid such as a coil, helix, clockwise and counter-clockwise extending braid, filar, wire or the like extending from the proximal mechanism portion 714 (corresponding to a proximal braided portion in the braid example) to a distal mechanism 716 (corresponding to a distal braided portion).

In one example, the expansion mechanism 712 extends proximally from the deformable distal mouth 710 to a proximal end portion 718 of the delivery catheter 708. In another example, the proximal mechanism portion 714 is coupled (e.g., by way of a butt weld, adhesive, bonding or the like) with an expansion catheter 720 provided within the delivery catheter 708. The expansion catheter 720 provides an operative interface between the expansion mechanism 712 and an operator (e.g., physician, clinician or the like) proximate the proximal end portion 718 of the delivery catheter 708.

The expansion mechanism 712 is movable relative to the delivery catheter 708 for instance, in one or more of longitudinal, rotational directions or the like. The distal mechanism portion 716 (in contrast to the remainder of the mechanism) is anchored to the deformable distal mouth 710. Accordingly, and as described further herein, with rotation or translation of the expansion mechanism 712, the mechanism 712 expands and correspondingly expands the deformable distal mouth 710 from a resting configuration (FIG. 7A) to an expanded configuration (FIG. 7B) to increase the profile of the deformable distal mouth 710 (to an expanded profile shown in FIG. 7B). The expanded configuration of the deformable distal mouth 710 facilitates reception of the balloon 706 within the delivery catheter 708.

The balloon system 700 is shown in the expanded configuration in FIG. 7B. As previously described, operation of the expansion mechanism 712 with the delivery catheter 708 enlarges the deformable distal mouth 710. Where the expansion mechanism 712 includes a braid, including but not limited to, a wire coil, filar coil, helical coil, helical filers, clockwise and counterclockwise coils (a braid) or the like, the rotation of the braid, for instance, in an unwinding direction correspondingly biases the expansion mechanism 712 outwardly for instance relative to the balloon catheter 704. Because the distal mechanism portion 716 (e.g., the distal braided portion) is anchored to the deformable distal mouth 710, the expansion of the expansion mechanism 712 biases the deformable distal mouth 710 outwardly into the expanded configuration (e.g., a trumpet configuration) sized and shaped to receive the balloon 706 therein.

In one example, the deformable distal mouth 710 is expanded, for instance, with a profile greater than a relaxed or deflated profile of the balloon 706. In another example, the deformable distal mouth 710 is provided in expanded configuration having a profile sufficiently large to receive the balloon 706 in a substantially inflated configuration (e.g., fully or partially deflated), and sliding movement of the balloon 706 into the delivery catheter 708 further contracts the balloon 706 and facilitates the continued withdrawal of the balloon 706 into the delivery catheter 708 for eventual retraction of the balloon system 700 from the subject.

As further shown in FIG. 7B and previously described herein, in one example, the balloon system 700 includes an expansion catheter 720 coupled with the expansion mechanism 712. The expansion catheter 720, like the expansion mechanism 712, is movable relative to the delivery catheter 708. Accordingly, one or more of rotation or longitudinal movement of the expansion catheter 720 relative to the delivery catheter 708 is transmitted to the expansion mechanism 712 and operates the expansion mechanism 712 to expand and accordingly also expand the deformable distal mouth 710 into the expanded configuration shown in FIG. 7B.

In one example, rotation of the expansion catheter 720 for instance in an unwinding direction of the expansion mechanism 712 correspondingly opens the expansion mechanism 712 and thereby also enlarges the deformable distal mouth 710 from the resting configuration to the expanded configuration. In another example, longitudinal movement of the expansion catheter 720 for instance in a distal direction (e.g., toward the distal mechanism portion 716) biases the expansion mechanism 712 in an outward manner because of the anchoring of the distal mechanism portion 716 to the deformable distal mouth 710.

After reception of the balloon 706 within the delivery catheter 708 in one example, one or more of the expansion mechanism 712 and the optional expansion catheter 720 are moved to reconfigure the deformable distal mouth 710 into the resting configuration shown, for instance, in FIG. 7A. The delivery catheter 708 with the balloon 806 therein is withdrawn from the vasculature, cavities or the like of the subject.

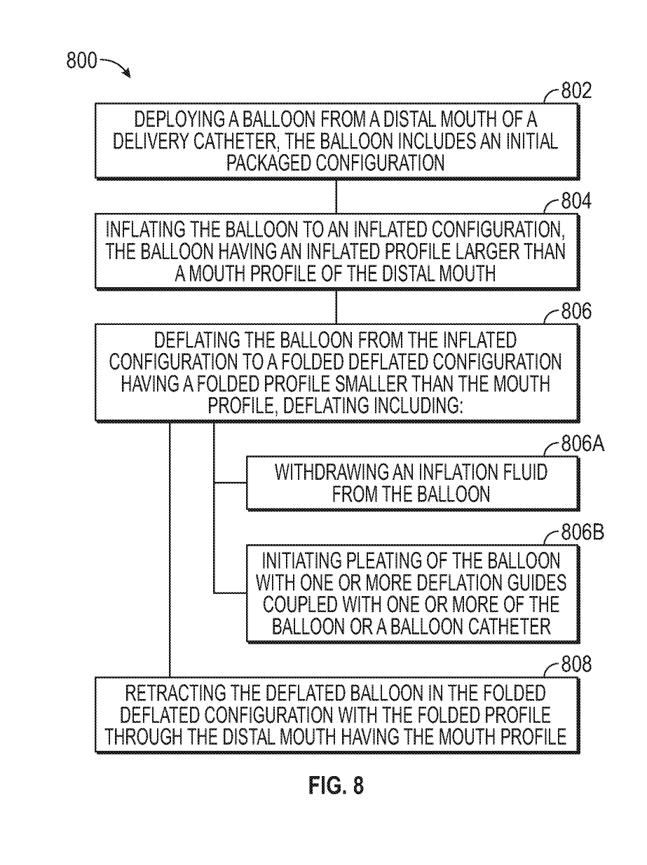

FIG. 8 shows one example of a method 800 for using a balloon system (one or more of the balloon systems described herein). In describing the method 800 reference is made to one or more components, features, functions, steps or the like described herein. For convenience, reference is made to the components, features, functions, steps and the like with reference numerals. Reference numerals provided are exemplary and are not exclusive. For instance, the features, components, functions, steps and the like described in the method 800 include, but are not limited to, the corresponding numbered elements, other corresponding features described herein, both numbered and unnumbered, as well as their equivalents.

At 802, the method 800 includes deploying a balloon assembly such as the balloon assembly 201 (shown in FIG. 3A) from a distal mouth 314 of delivery catheter 206. The balloon assembly 201 includes a balloon 204 in an initial package configuration (with the balloon 204 folded and having a profile that fits within the delivery lumen 312 of the delivery catheter 206). At 804, the balloon 204 is inflated to an inflated configuration (e.g., shown in FIG. 3A). In one example, the balloon 204 has an inflated profile larger than the distal mouth 114 (a mouth profile 116). Similarly, the balloon 204 in the inflated configuration shown in FIG. 3A has a larger profile than the mouth profile of the distal mouth 314.

At 806, the method 800 includes deflating the balloon 204 from the inflated configuration (shown in FIG. 3A) through a folded deflated configuration (shown in FIG. 3C) having a folded profile 322 smaller than the mouth profile of the distal mouth 314. In one example, deflating the balloon 204 includes at 806A withdrawing an inflation fluid from the balloon 204, for instance by way of the inflation lumen 310 of the balloon catheter 202. As described herein deflating the balloon 204 further includes at 806B initiating pleating (including pleats, creases, ribs, ruffles or the like) of the balloon 204 with one or more deflation guides 304 coupled with one or more of the balloon 204 or the balloon catheter 202. The one or more deflation guides include but are not limited to, one or more of deflation struts, a deflation shell, a telescoping shaft, or the like. As described herein, the one or more deflation guides predispose the balloon 204 to pleat (e.g., initiate, bias or the like) and guide folding of the balloon 204 into the folded deflated configuration.

At 808, the method 800 further includes retracting the deflated balloon 204 in the folded deflated configuration (shown in FIG. 3C) with the minimized folded profile 322 (relative to a flat balloon profile 112 shown in FIG. 1C) through the distal mouth 314 and into the delivery catheter 206.

Several options for the method 800 follow. In one example inflating the balloon 204 includes stretching the one or more deflation guides 304, for instance elastomer or compliant deflation guides 304 or the like from a relaxed configuration to a stretched configuration. In another example, stretching the one or more deflation guides 304 from the relaxed configuration to the stretched configuration includes stretching the one or more deflation guides proximate to when the balloon assumes the inflated profile. For instance, in one example, the deflation guides are coupled with the balloon 204 in an example and configured for slidable movement over at least a portion of the balloon 204. Accordingly with inflation of the balloon, the deflation guides are in one example dimensioned and configured to slide over the balloon 204 and stretch upon full inflation (when the guides are taut, and just before full inflation) of the balloon 204 into the inflated configuration. Accordingly, in one example the compressive force supplied by the deflation guides 304 is minimized relative to the inflated balloon 204 to accordingly facilitate the full deployment of the balloon into the desired inflated shape (e.g., without escalating inflation pressure).

In yet another example, initiating pleating of the balloon 204 includes transitioning the one or more deflation guides 304 from the stretched configuration to the relaxed configuration. For instance, as the balloon 204 begins to deflate in one example, the stretched deflation guides 304 apply compressive force according to tension in the stretched guides and then relax and arrest further application of the compressive force. Accordingly, in one example, the balloon 204 is provided in a pleated guiding configuration (e.g., with pleats 316) with operation of the deflation guides 304, and further deflation of the balloon 204 (by the withdrawal of inflation fluid) completes deflation of the balloon 204 into the folded deflated configuration. The deflation guides 304, having initiated pleating, guide the folding of the balloon 204 into the folded deflated configuration including the fold 318 shown in FIG. 3C. The folded deflated configuration includes the minimized folded profile 322 less than the mouth profile of the distal mouth 314.

The method 800 includes in another example, distributed and local application of compressive forces to the balloon 204 to initiate pleating of the balloon 204. For instance, initiating pleating of the balloon 204 includes applying a compressive force at one or more locations on the balloon 204 with the one or more deflation guides 304. In another example, applying the compressive force at one or more locations on the balloon 204 includes applying the compressive force continuously around the balloon with one or more deflation guides, for instance the deflation shells, a plurality of deflation struts arranged around the balloon or the like described herein. In another example, applying the compressive force at one or more locations includes applying the compressive force at discrete locations around the balloon, for instance, with one or more deflation guides 304 such as the one or more deflation struts described herein distributed (spaced) around the balloon 204.

As further described herein, in one example, the deflation guides include a telescoping shaft 610 received within an exterior shaft, such as the balloon catheter 602 of the balloon assembly or systems as described herein. The telescoping shaft 610 (shown in FIGS. 6A, 6B) is moved, for instance by one or more of longitudinal or rotational movement, of the shaft 610 relative to the balloon catheter 602 to initiate pleating of the balloon into the pleated guiding configuration, for instance shown in FIG. 3B.

In another example, the method 800 includes expanding the distal mouth, for instance the deformable distal mouth 710 show, in FIG. 7A from a resting configuration to an expanded configuration with an expansion mechanism such as the expansion mechanism 712. Expanding of the deformable distal mouth 710 in one example includes transitioning the deformable distal mouth from an initial resting configuration having a mouth profile shown in FIG. 7A (e.g., corresponding to the cross sectional area at the deformable distal mouth the width, radius, diameter of the distal mouth or the like) to the expanded configuration having the enlarged mouth profile shown in FIG. 7B.

In one example, expanding the distal mouth 710 includes one or more of rotating or translating a portion of the expansion mechanism 712 relative to a delivery catheter 708. For instance, expanding the deformable distal mouth 712 includes one or more of rotating or translating a portion of the expansion mechanism 712 including but not limited to, a braid (e.g., a wire or filar that is helically wound, coiled, a clockwise and counterclockwise extending braid or the like) relative to a catheter such as the delivery catheter 708 shown in FIG. 7A. In one example, a distal mechanism portion 716 (e.g., distal braid portion) of the expansion mechanism 712 is anchored to the delivery catheter 708 proximate to the deformable distal mouth 710. The remainder of the expansion mechanism 712 is movable relative to the delivery catheter 708. One or more of longitudinal or rotational movement of the expansion mechanism 712 expands mechanism at the distal mechanism portion 716 and accordingly expands the deformable distal mouth 710 (as shown in FIG. 7B).

In another example, and as previously described herein, the proximal mechanism portion 714 is provided at the proximal end portion of the delivery catheter 718. In still another example, the proximal mechanism portion is coupled with an expansion catheter 720. The expansion mechanism 712 extending from the proximal mechanism portion 714 to the deformable distal mouth 710 is translated or rotated itself or with the expansion catheter 720 to transition the deformable distal mouth 710 from the resting configuration shown in FIG. 7A to the expanded configuration shown in FIG. 7B having the enlarged mouth profile. The expanded mouth profile facilitates the reception of the balloon 706 within the delivery catheter 708 and in one example facilitates the reception of an inflated (partially or fully deflated) balloon 706 therein.

In examples of the subject matter disclosed herein, any portion of the balloon system (e.g., the balloon, balloon catheter or the like)includes a coating, such as a hydrophilic lubricious coating. For example, hydrophilic polymeric base coatings are applied to portions of the balloon system to impart lubricity and decrease friction. In other embodiments, a portion (a part or the entirety) of an insertion tool or article, or any portions of catheters of the disclosure are associated with a low friction article, such as a Teflon sleeve. In some embodiments, all or a portion of the inner diameter of the catheter (e.g., the balloon catheter, delivery catheter or the like) is coated with a hydrophilic coating, lined with a lubricious low friction sleeve (e.g. PTFE and PTFE liners) or the like. In some embodiments, all or a portion of the outer surface of the balloon catheter is coated with a hydrophilic coating, lined with a lubricious low friction sleeve or the like. Other materials for providing a lubricious low friction coating include, but are not limited to, a silicone oil, perfluorinated oils and waxes, optionally with covalent bonding, to decrease friction. These examples of low friction and hydrophilic coatings assist in increasing lubricity and decreasing the loss of bioactive agents (e.g., coatings) and the generation of particulate.

One class of hydrophilic polymers useful as polymeric materials for hydrophilic base coat formation includes synthetic hydrophilic polymers. Synthetic hydrophilic polymers that are biostable (i.e., that show no appreciable degradation in vivo) are prepared from a suitable monomer including, but not limited to, acrylic monomers, vinyl monomers, ether monomers, or combinations of any one or more of these types of monomers. Acrylic monomers include, but are not limited to, methacrylate, methyl methacrylate, hydroxyethyl methacrylate, hydroxyethyl acrylate, methacrylic acid, acrylic acid, glycerol acrylate, glycerol methacrylate, acrylamide, methacrylamide, dimethylacrylamide (DMA), and one or more of derivatives or mixtures of any of these. Vinyl monomers include, but are not limited to, vinyl acetate, vinylpyrrolidone, vinyl alcohol, and derivatives of any of these. Ether monomers include, but are not limited to, ethylene oxide, propylene oxide, butylene oxide, and derivatives of any of these. Examples of polymers formed from these monomers include, but are not limited to, poly(acrylamide), poly(methacrylamide), poly(vinylpyrrolidone), poly(acrylic acid), poly(ethylene glycol), poly(vinyl alcohol), and poly(HEMA). Examples of hydrophilic copolymers include, but are not limited to, methyl vinyl ether/maleic anhydride copolymers and vinyl pyrrolidone/(meth)acrylamide copolymers. Further, mixtures of one or more or homopolymers or copolymers are used in some examples for hydrophilic base coats.

Examples of some acrylamide-based polymers, such as poly(N,Ndimethylacrylamide-co-aminopropylmethacrylamide) and poly(acrylamide-co-N,Ndimethylaminopropylmethacrylamide) are described in example 2 of U.S. Pat. No. 7,807,750 (Taton et al.), the disclosure of which is incorporated herein by reference.

Other hydrophilic polymers used with the subject matter of this disclosure include derivatives of acrylamide polymers with photoreactive groups. One such representative hydrophilic polymer is the copolymerization of N-[3-(4-benzoylbenzamido)propyl] methacrylamide (Formula I) with N-(3-aminopropyl)methacrylamide (Formula II) to produce the polymer poly(N-3-aminopropyl)methacrylamide-co-N-[3-(4-benzoylbenzamido)propyl]methacrylamide (Formula III). The preparation of the polymer is disclosed in Example 1 of US Patent Publication 2007/0032882 (to Lodhi, et al.), the full content of which is incorporated herein by reference.

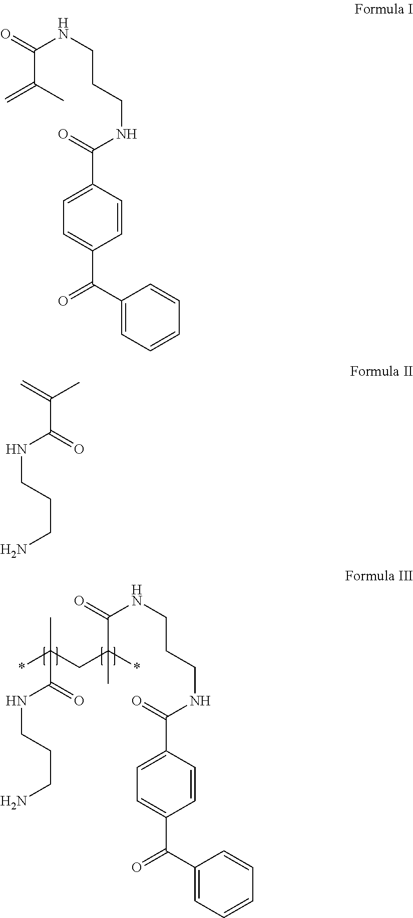

In some embodiments, the hydrophilic polymer includes a vinyl pyrrolidone polymer, or a vinyl pyrrolidone/(meth)acrylamide copolymer such as poly(vinylpyrrolidone-co-methacrylamide). If a PVP copolymer is used, in some examples it includes a copolymer of vinylpyrrolidone and a monomer selected from the group of acrylamide monomers. Exemplary acrylamide monomers include (meth)acrylamide and (meth)acrylamide derivatives, such as alkyl (meth)acrylamide, as exemplified by dimethylacrylamide, and aminoalkyl (meth)acrylamide, as exemplified by aminopropylmethacrylamide and dimethylaminopropylmethacrylamide. For example, poly(vinylpyrrolidone-co-N,N-dimethylaminopropylmethacrylamide) is described in example 2 of U.S. Pat. No. 7,807,750 (Taton et al.).

In one embodiment, the polymers and copolymers as described are derivatized with one or more photoactivatable group(s). Exemplary photoreactive groups that can be pendent from biostable hydrophilic polymer include aryl ketones, such as acetophenone, benzophenone, anthraquinone, anthrone, quinone, and anthrone-like heterocycles. Aryl ketones herein can specifically include diaryl ketones. Polymers herein can provide a hydrophilic polymer having a pendent activatable photogroup that can be applied to the expandable and collapsible structure, and can then treated with actinic radiation sufficient to activate the photogroups and cause covalent bonding to a target, such as the material of the expandable and collapsible structure. Use of photo-hydrophilic polymers can be used to provide a durable coating of a flexible hydrogel matrix, with the hydrophilic polymeric materials covalently bonded to the material of the expandable and collapsible structure.

A hydrophilic polymer having pendent photoreactive groups can be used to prepare the flexible hydrogel coating. Methods of preparing hydrophilic polymers having photoreactive groups are known in the art. For example, methods for the preparation of photo-PVP are described in U.S. Pat. No. 5,414,075 (to Swan et al), the disclosure of which is incorporated herein by reference. Hydrophilic photo-polyacrylamide polymers such as poly(acrylamide-co-N-(3-(4-benzoylbenzamido)propyl) methacylamide), “Photo PA”, and derivatives thereof can be used to form hydrophilic base coats in exemplary embodiments of the present disclosure. Methods for the preparation of photo-polyacrylamide are described in U.S. Pat. No. 6,007,833 (to Chudzik et al.), the disclosure of which is incorporated herein by reference.

Other embodiments of hydrophilic base coats include derivatives of photo-polyacrylamide polymers incorporating additional reactive moieties. Some exemplary reactive moieties include N-oxysuccinimide and glycidyl methacrylate. Representative photo-polyacrylamide derivatives incorporating additional reactive moieties include poly(acrylamide-co-maleic-6-aminocaproic acid-N-oxysuccinimide-co-N-(3-(4-benzoylbenzamido)propyl)methacrylamide) and poly(acrylamide-co-(3-(4-benzoylbenzamido)propyl)methacrylamide)-co-glycidylmethacrylate. Additional photo-polyacrylamide polymers incorporating reactive moieties are described in U.S. Pat. No. 6,465,178 (to Chappa, et al.), U.S. Pat. No. 6,762,019 (to Swan, et al.) and U.S. Pat. No. 7,309,593 (to Ofstead, et al.), the disclosures of which are herein incorporated by reference.

Other embodiments of exemplary hydrophilic base coats that include derivatives of photo-polyacrylamide polymers incorporating additional reactive moieties can be found in U.S. Pat. No. 6,514,734 (to Clapper, et al.), the disclosure of which is incorporated herein by reference in its entirety.

In yet other embodiments, the hydrophilic base coat can include derivatives of photo-polyacrylamide polymers incorporating charged moieties. Charged moieties include both positively and negatively charged species. Exemplary charged species include, but are not limited to, sulfonates, phosphates and quaternary amine derivatives. Some examples include the negatively charged species N-acetylated poly(acrylamide-co-sodium-2-acrylamido-2-methylpropanesulfonate-co-N-(3-(4-benzoylbenzamido)propyl)methacrylamide)-co-methoxy poly(ethylene glycol) monomethacrylate. Other negatively charged species that can be incorporated into the hydrophilic base coat are described in U.S. Pat. No. 4,973,493 (to Guire et al.), the disclosure of which is incorporated herein by reference in its entirety. Positively charged species can include poly(acrylamide-co-N-(3-(4-benzoylbenzamido)propyl)methacrylamide) -co-(3-(methacryloylamino)propyl)trimethylammonium chloride. Other positively charged species that can be incorporated into the hydrophilic base coat are described in U.S. Pat. No. 5,858,653 (to Duran et al.), the disclosure of which is incorporated herein by reference in its entirety.

In another embodiment, the polymers and copolymers as described are derivatized with one or more polymerizable group(s). Polymers with pendent polymerizable groups are commonly referred to as macromers. The polymerizable group(s) can be present at the terminal portions (ends) of the polymeric strand or can be present along the length of the polymer. In one embodiment polymerizable groups are located randomly along the length of the polymer.

Exemplary hydrophilic polymer coatings can be prepared using polymer grafting techniques. Polymer grafting techniques can include applying a nonpolymeric grafting agent and monomers to a substrate surface then causing polymerization of the monomers on the substrate surface upon appropriate activation (for example, but not limited to, UV radiation) of the grafting agent. Grafting methods producing hydrophilic polymeric surfaces are exemplified in U.S. Pat. Nos. 7,348,055; 7,736,689 and 8,039,524 (all to Chappa et al.) the full disclosures of which are incorporated herein by reference.

Optionally, the coating can include a crosslinking agent. A crosslinking agent can promote the association of polymers in the coating, or the bonding of polymers to the coated surface. The choice of a particular crosslinking agent can depend on the ingredients of the coating composition.

Suitable crosslinking agents can include two or more activatable groups, which can react with the polymers in the composition. Suitable activatable groups can include photoreactive groups as described herein, like aryl ketones, such as acetophenone, benzophenone, anthraquinone, anthrone, quinone, and anthrone-like heterocycles. A crosslinking agent including a photoreactive group can be referred to as a photo-crosslinker or photoactivatable crosslinking agent. The photoactivatable crosslinking agent can be ionic, and can have good solubility in an aqueous composition. Thus, in some embodiments, at least one ionic photoactivatable crosslinking agent can be used to form the coating. The ionic crosslinking agent can include an acidic group or salt thereof, such as selected from sulfonic acids, carboxylic acids, phosphonic acids, salts thereof, and the like. Exemplary counter ions include alkali, alkaline earths metals, ammonium, protonated amines, and the like.

Exemplary ionic photoactivatable crosslinking agents include 4,5-bis(4-benzoylphenylmethyleneoxy) benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,077,698 (Swan et al.), U.S. Pat. No. 6,278,018 (Swan), U.S. Pat. No. 6,603,040 (Swan) and U.S. Pat. No. 7,138,541 (Swan) the disclosures of which are incorporated herein by reference.

Other exemplary ionic photoactivatable crosslinking agents include ethylenebis(4-benzoylbenzyldimethylammonium) dibromide and hexamethylenebis(4-benzoylbenzyldimethylammonium) dibromide and the like. See U.S. Pat. No. 5,714,360 (Swan et al.) the disclosures of which are incorporated herein by reference.

In yet other embodiments, restrained multifunctional reagents with photoactivable crosslinking groups can be used. In some examples these restrained multifunctional reagents include tetrakis (4-benzoylbenzyl ether) of pentaerthyritol and the tetrakis (4-benzoylbenzoate ester) of pentaerthyritol. See U.S. Pat. No. 5,414,075 (Swan et al.) and U.S. Pat. No. 5,637,460 (Swan et al.) the disclosures of which are incorporated herein by reference.

Additional crosslinking agents can include those having formula Photo1-LG-Photo2, wherein Photo1 and Photo2 independently represent at least one photoreactive group and LG represents a linking group comprising at least one silicon or at least one phosphorus atom, wherein the degradable linking agent comprises a covalent linkage between at least one photoreactive group and the linking group, wherein the covalent linkage between at least one photoreactive group and the linking group is interrupted by at least one heteroatom. See U.S. Pat. No. 8,889,760 (Kurdyumov, et al.), the disclosure of which is incorporated herein by reference. Further crosslinking agents can include those having a core molecule with one or more charged groups and one or more photoreactive groups covalently attached to the core molecule by one or more degradable linkers. See U.S. Publ. Pat. App. No. 2011/0144373 (Swan, et al.), the disclosure of which is incorporated herein by reference.

In some embodiments, the first and/or second crosslinking agent can have a molecular weight of less than about 1500 kDa. In some embodiments the crosslinking agent can have a molecular weight of less than about 1200, 1100, 1000, 900, 800, 700, 600, 500, or 400.

In some embodiments, at least one of the first and second crosslinking agents comprising a linking agent having formula Photo1-LG-Photo2, wherein Photo1 and Photo2, independently represent at least one photoreactive group and LG represents a linking group comprising at least one silicon or at least one phosphorus atom, there is a covalent linkage between at least one photoreactive group and the linking group, wherein the covalent linkage between at least one photoreactive group and the linking group is interrupted by at least one heteroatom.

In some embodiments, at least one of the first and second crosslinking agents comprising a linking agent having a formula selected from:

(a)

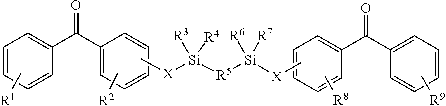

wherein R1, R2, R8 and R9 are any substitution; R3, R4, R6 and R7 are alkyl, aryl, or a combination thereof; R5 is any substitution; and each X, independently, is O, N, Se, S, or alkyl, or a combination thereof;

(b)

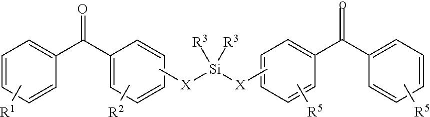

wherein R1 and R5 are any substitution; R2 and R4 can be any substitution, except OH; R3 can be alkyl, aryl, or a combination thereof; and X, independently, are O, N, Se, S, alkylene, or a combination thereof;
(c)

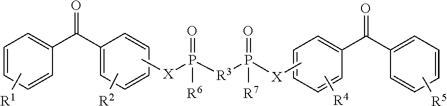

wherein R1, R2, R4 and R5 are any substitution; R3 is any substitution; R6 and R7 are alkyl, aryl, or a combination thereof; and each X can independently be O, N, Se, S, alkylene, or a combination thereof; and
(d)

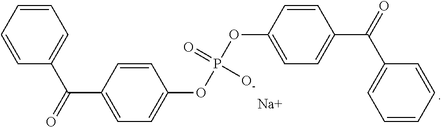

In a particular embodiment, the crosslinking agent can be bis(4-benzoylphenyl) phosphate.

In some embodiments, the photoactivatable crosslinking agent can be ionic, and can have good solubility in an aqueous composition, such as the first and/or second coating composition. Thus, in some embodiments, at least one ionic photoactivatable crosslinking agent is used to form the coating. In some cases, an ionic photoactivatable crosslinking agent can crosslink the polymers within the second coating layer which can also improve the durability of the coating.

Any suitable ionic photoactivatable crosslinking agent can be used. In some embodiments, the ionic photoactivatable crosslinking agent is a compound of formula I: X1-Y—X2 where Y is a radical containing at least one acidic group, basic group, or a salt of an acidic group or basic group. X1 and X2 are each independently a radical containing a latent photoreactive group. The photoreactive groups can be the same as those described herein. Spacers can also be part of X1 or X2 along with the latent photoreactive group. In some embodiments, the latent photoreactive group includes an aryl ketone or a quinone.

The radical Y in formula I provides the desired water solubility for the ionic photoactivatable crosslinking agent. The water solubility (at room temperature and optimal pH) is at least about 0.05 mg/ml. In some embodiments, the solubility is about 0.1 to about 10 mg/ml or about 1 to about 5 mg/ml.

In some embodiments of formula I, Y is a radical containing at least one acidic group or salt thereof. Such a photoactivatable crosslinking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic crosslinking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X1 and X2 can contain photoreactive groups such as aryl ketones. Such compounds include 4,5-bis(4-benzoylphenylmethyleneoxy) benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018 (to Swan). The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate. For example, compounds of formula I can have a Y radical that contains an ammonium group; X1 and X2 can contain photoreactive groups that include aryl ketones. Such photoactivatable crosslinking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl) hexamethylenetetraminediium salt, bis[2-(4-benzoylbenzyldimethylammonio) ethyl]-4-benzoylbenzylmethylammonium salt; 4,4-bis(4-benzoylbenzyl) morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl) piperzinediium salt. See U.S. Pat. No. 5,714,360 (to Swan et al.). The counter ion is typically a carboxylate ion or a halide. On one embodiment, the halide is bromide.

In other embodiments, the ionic photoactivatable crosslinking agent can be a compound having the formula:

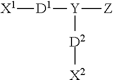

wherein X1 includes a first photoreactive group; X2 includes a second photoreactive group; Y includes a core molecule; Z includes at least one charged group; D1 includes a first degradable linker; and D2 includes a second degradable linker. Additional exemplary degradable ionic photoactivatable crosslinking agents are described in US Patent Application Publication US 2011/0144373 (Swan et al., “Water Soluble Degradable Crosslinker”), the disclosure of which is incorporated herein by reference.

In some aspects a non-ionic photoactivatable crosslinking agent can be used. In one embodiment, the non-ionic photoactivatable crosslinking agent has the formula XR1R2R3R4, where X is a chemical backbone, and R1, R2, R3, and R4 are radicals that include a latent photoreactive group. Exemplary non-ionic crosslinking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

In other embodiments, the non-ionic photoactivatable crosslinking agent can be represented by the formula:

PG2-LE2-X-LE1-PG1

wherein PG1 and PG2 include, independently, one or more photoreactive groups, for example, an aryl ketone photoreactive group, including, but not limited to, aryl ketones such as acetophenone, benzophenone, anthraquinone, anthrone, anthrone-like heterocycles, their substituted derivatives or a combination thereof; LE1 and LE2 are, independently, linking elements, including, for example, segments that include urea, carbamate, or a combination thereof; and X represents a core molecule, which can be either polymeric or non-polymeric, including, but not limited to a hydrocarbon, including a hydrocarbon that is linear, branched, cyclic, or a combination thereof; aromatic, non-aromatic, or a combination thereof; monocyclic, polycyclic, carbocyclic, heterocyclic, or a combination thereof; benzene or a derivative thereof; or a combination thereof. Other non-ionic crosslinking agents are described, for example, in Publ. No. US 2012/0149934(to Kurdyumov, “Photocrosslinker”), the disclosure of which is incorporated herein by reference.

Further embodiments of non-ionic photoactivatable crosslinking agents can include, for example, those described in US Pat. Publication 2013/0143056 (Swan et al., “Photo-Vinyl Linking Agents”), the disclosure of which is incorporated herein by reference. Exemplary crosslinking agents can include non-ionic photoactivatable crosslinking agents having the general formula R1-X—R2, wherein R1 is a radical comprising a vinyl group, X is a radical comprising from about one to about twenty carbon atoms, and R2 is a radical comprising a photoreactive group.

A single photoactivatable crosslinking agent or any combination of photoactivatable crosslinking agents can be used in forming the coating. In some embodiments, at least one nonionic crosslinking agent such as tetrakis(4-benzoylbenzyl ether) of pentaerythritol can be used with at least one ionic crosslinking agent. For example, at least one non-ionic photoactivatable crosslinking agent can be used with at least one cationic photoactivatable crosslinking agent such as an ethylenebis(4-benzoylbenzyldimethylammonium) salt or at least one anionic photoactivatable crosslinking agent such as 4,5-bis(4-benzoyl-phenylmethyleneoxy)benzene-1,3-disulfonic acid or salt. In another example, at least one nonionic crosslinking agent can be used with at least one cationic crosslinking agent and at least one anionic crosslinking agent. In yet another example, a least one cationic crosslinking agent can be used with at least one anionic crosslinking agent but without a non-ionic crosslinking agent. An exemplary crosslinking agent is disodium 4,5-bis[(4-benzoylbenzyl)oxy]-1,3-benzenedisulfonate (DBDS). This reagent can be prepared by combining 4,5-Dihydroxylbenzyl-1,3-disulfonate (CHBDS) with 4-bromomethylbenzophenone (BMBP) in THF and sodium hydroxide, then refluxing and cooling the mixture followed by purification and recrystallization (also as described in U.S. Pat. No. 5,714,360, incorporated herein by reference).

Further crosslinking agents can include the crosslinking agents described in U.S. Pat. No. 8,487,137 (to Guire et al.) and U.S. Pat. No. 7,772,393 (to Guire et al.) the content of all of which is herein incorporated by reference.

In some embodiments, crosslinking agents can include boron-containing linking agents including, but not limited to, the boron-containing linking agents disclosed in U.S. Pat. No. 9,410,044 (to Kurdyumov) the content of which is herein incorporated by reference. By way of example, linking agents can include borate, borazine, or boronate groups and coatings and devices that incorporate such linking agents, along with related methods. In an embodiment, the linking agent includes a compound having the structure (I):

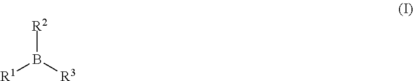

wherein R1 is a radical comprising a photoreactive group; R2 is selected from OH and a radical comprising a photoreactive group, an alkyl group and an aryl group; and R3 is selected from OH and a radical comprising a photoreactive group. In some embodiments the bonds B—R1, B—R2 and B—R3 can be chosen independently to be interrupted by a heteroatom, such as O, N, S, or mixtures thereof.

Additional agents for use with embodiments herein can include stilbene-based reactive compounds including, but not limited to, those disclosed in U.S. Pat. No. 8,487,137, entitled “Stilbene-Based Reactive Compounds, Polymeric Matrices Formed Therefrom, and Articles Visualizable by Fluorescence” by Kurdyumov et al., the content of which is herein incorporated by reference.

Additional photoreactive agents, crosslinking agents, hydrophilic coatings, and associated reagents are disclosed in U.S. Pat. No. 8,513,320 (to Rooijmans et al.); 8,809,411 (to Rooijmans); and 2010/0198168 (to Rooijmans), the content of all of which is herein incorporated by reference.

Natural polymers can also be used to form the hydrophilic base coat. Natural polymers include polysaccharides, for example, polydextrans, carboxymethylcellulose, and hydroxymethylcellulose; glycosaminoglycans, for example, hyaluronic acid; polypeptides, for example, soluble proteins such as collagen, albumin, and avidin; and combinations of these natural polymers. Combinations of natural and synthetic polymers can also be used.

In some instances a tie layer can be used to form the hydrophilic base layer. In yet other instances the tie layer can be added to the hydrophilic base layer. The tie layer can act to increase the adhesion of the hydrophilic base layer to the substrate. In other embodiments, the tie layer can act to increase adhesion of the hydrophobic active agent to the hydrophilic base layer. Exemplary ties layers include, but are not limited to silane, butadiene, polyurethane and parylene. Silane tie layers are described in US Patent Publication 2012/0148852 (to Jelle, et al.), the content of which is herein incorporated by reference.

In exemplary embodiments, the hydrophilic base layer can include tannic acid, polydopamine or other catechol containing materials.

In some embodiments of the present disclosure medicaments (e.g., bioactive agents) can be coated on balloon catheters. Additionally, excipients can be coated on balloon catheters to provide improved in vivo transfer characteristics of medicaments. Materials and devices for delivery of medicaments are described in U.S. Pat. Publications 2015/0140107 and 2012-0296274 (both to Slager), the content of both of which are herein incorporated by reference.

### VARIOUS NOTES & EXAMPLES

Example 1 can include subject matter such as a balloon system comprising: a balloon catheter extending from a proximal end portion to a distal end portion, the balloon catheter includes an inflation lumen therein; and a balloon assembly coupled with the distal end portion of the balloon catheter, the balloon assembly includes: a balloon having distal and proximal balloon ends, the balloon includes inflated and folded deflated configurations, and one or more deflation guides coupled with the balloon, the one or more deflation guides are configured to bias the balloon toward the folded deflated configuration.

Example 2 can include, or can optionally be combined with the subject matter of Example 1, to optionally include wherein the one or more deflation guides includes a plurality of deflation guides coupled along the balloon.

Example 3 can include, or can optionally be combined with the subject matter of one or any combination of Examples 1 or 2 to optionally include wherein the one or more deflation guides are within the balloon.

Example 4 can include, or can optionally be combined with the subject matter of one or any combination of Examples 1-3 to optionally include wherein the one or more deflation guides include at least one deflation strut extending between the proximal and distal balloon ends.

Example 5 can include, or can optionally be combined with the subject matter of one or any combination of Examples 1-4 to optionally include wherein the at least one deflation strut includes a plurality of deflation struts coupled at distributed locations around the balloon.

Example 6 can include, or can optionally be combined with the subject matter of Examples 1-5 to optionally include wherein the at least one deflation strut includes a deflation strut cage coupled around the balloon.

Example 7 can include, or can optionally be combined with the subject matter of Examples 1-6 to optionally include wherein the one or more deflation guides include a deflation shell continuously coupled across the balloon.

Example 8 can include, or can optionally be combined with the subject matter of Examples 1-7 to optionally include wherein the deflation shell is continuously coupled across one or more of a balloon interior or exterior.

Example 9 can include, or can optionally be combined with the subject matter of Examples 1-8 to optionally include wherein the deflation shell is coupled between a balloon interior and a balloon exterior.

Example 10 can include, or can optionally be combined with the subject matter of Examples 1-9 to optionally include wherein the balloon has a first compliance, and the one or more deflation guides have a second compliance greater than the first compliance.

Example 11 can include, or can optionally be combined with the subject matter of Examples 1-10 to optionally include wherein the balloon is non-compliant and the one or more deflation guides include an elastomer having a greater compliance than the balloon.

Example 12 can include, or can optionally be combined with the subject matter of Examples 1-11 to optionally include wherein the one or more deflation guides are configured to provide a compressive force at one or more discrete locations around the balloon to bias the balloon toward the folded deflated configuration.

Example 13 can include, or can optionally be combined with the subject matter of Examples 1-12 to optionally include wherein the one or more deflation guides are configured to initiate a pleated guiding configuration in the balloon that guides folding of the balloon to the folded deflated configuration.

Example 14 can include, or can optionally be combined with the subject matter of Examples 1-13 to optionally include wherein the one or more deflation guides are configured to initiate the pleated guiding configuration immediately after the balloon begins deflation.

Example 15 can include, or can optionally be combined with the subject matter of Examples 1-14 to optionally include wherein the one or more deflation guides are configured to arrest bias of the balloon at the pleated guiding configuration of the balloon.

Example 16 can include, or can optionally be combined with the subject matter of Examples 1-15 to optionally include wherein the balloon catheter and the balloon assembly include the one or more deflation guides.

Example 17 can include, or can optionally be combined with the subject matter of Examples 1-16 to optionally include wherein the balloon catheter includes an exterior shaft and the one or more deformation guides include a telescoping interior shaft within the exterior shaft, the exterior shaft is coupled with the proximal balloon end, and the interior shaft is coupled with the distal balloon end, the interior shaft is slidable within the exterior shaft, and one or more of relative rotation or longitudinal relative movement of the interior shaft relative to the exterior shaft is configured to bias the balloon toward the folded deflated configuration.

Example 18 can include, or can optionally be combined with the subject matter of Examples 1-17 to optionally include a delivery catheter, the balloon assembly and the balloon catheter are slidable within a delivery lumen of the delivery catheter, and the delivery catheter includes: a deformable distal mouth including a resting configuration and an expanded configuration, and an expansion mechanism coupled with the deformable distal mouth, the expansion mechanism is configured to transition the deformable distal mouth between the resting and expanded configurations.

Example 19 can include, or can optionally be combined with the subject matter of Examples 1-18 to optionally include wherein the expansion mechanism includes a braid having proximal and distal braid portions, the distal braid portion is anchored to the deformable distal mouth, and the braid transitions the deformable distal mouth with one or more of rotation or longitudinal translation of the braid relative to the delivery catheter.

Example 20 can include, or can optionally be combined with the subject matter of Examples 1-19 to optionally include a balloon system comprising: a balloon catheter extending from a proximal end portion to a distal end portion, the balloon catheter includes an inflation lumen therein; a balloon assembly coupled with the distal end portion of the balloon catheter, the balloon assembly includes: a balloon having distal and proximal balloon ends, the balloon includes inflated and folded deflated configurations, and one or more elastomer deflation struts coupled with the balloon between the distal and proximal balloon ends; and wherein the one or more elastomer deflation struts are configured to stretch in the inflated configuration of the balloon and to initiate pleating of the balloon with transition from the inflated configuration toward the folded deflated configuration according to the stretching of the one or more elastomer deflation struts.

Example 21 can include, or can optionally be combined with the subject matter of Examples 1-20 to optionally include wherein the one or more elastomer deflation struts are within the balloon.

Example 22 can include, or can optionally be combined with the subject matter of Examples 1-21 to optionally include wherein the one or more elastomer deflation struts are coupled along the balloon.

Example 23 can include, or can optionally be combined with the subject matter of Examples 1-22 to optionally include wherein at least a portion of each of the one or more elastomer deflation struts are slidably coupled along the balloon.

Example 24 can include, or can optionally be combined with the subject matter of Examples 1-23 to optionally include wherein at least a portion of each of the one or more elastomer deflation struts is anchored along the balloon.

Example 25 can include, or can optionally be combined with the subject matter of Examples 1-24 to optionally include wherein the one or more elastomer deflation struts are anchored to a midpoint of the balloon.

Example 26 can include, or can optionally be combined with the subject matter of Examples 1-25 to optionally include wherein the one or more elastomer deflation struts include a plurality of elastomer deflation struts coupled at distributed locations around the balloon.

Example 27 can include, or can optionally be combined with the subject matter of Examples 1-26 to optionally include wherein the plurality of elastomer deflation struts are arranged as a deflation cage around the balloon.

Example 28 can include, or can optionally be combined with the subject matter of Examples 1-27 to optionally include wherein the one or more elastomer deflation struts include at least one helical deflation strut extending around the balloon between the proximal and distal balloon ends.

Example 29 can include, or can optionally be combined with the subject matter of Examples 1-28 to optionally include wherein the one or more elastomer deflation struts include one or more deflation rings extending around the balloon.

Example 30 can include, or can optionally be combined with the subject matter of Examples 1-29 to optionally include wherein the balloon has a first compliance and the one or more elastomer deflation struts have a second compliance greater than the first compliance.

Example 31 can include, or can optionally be combined with the subject matter of Examples 1-30 to optionally include wherein the one or more elastomer deflation struts are configured to provide a compressive force at one or more discrete locations around the balloon according to the stretching of the one or more elastomer deflation struts.

Example 32 can include, or can optionally be combined with the subject matter of Examples 1-31 to optionally include wherein the one or more elastomer deflation struts include stretched and relaxed configurations: in the inflated configuration of the balloon the one or more elastomer deflation struts are in the stretched configuration, and in the folded deflated configuration the one or more elastomer deflation struts are in the relaxed configuration.

Example 33 can include, or can optionally be combined with the subject matter of Examples 1-32 to optionally include wherein the one or more elastomer deflation struts are configured to transition from the stretched configuration to the relaxed configuration at initiating of pleating.

Example 34 can include, or can optionally be combined with the subject matter of Examples 1-33 to optionally include wherein the one or more elastomer deflation struts are configured to guide deflation of the balloon toward the folded deflated configuration with initiating of pleating of the balloon.

Example 35 can include, or can optionally be combined with the subject matter of Examples 1-34 to optionally include wherein the one or more elastomer deflation struts are configured to arrest bias of the balloon at the initiating of pleating in the balloon with relaxation of the one or more elastomer deflation struts.

Example 36 can include, or can optionally be combined with the subject matter of Examples 1-35 to optionally include a balloon system comprising: a balloon catheter extending from a proximal end portion to a distal end portion, the balloon catheter includes an inflation lumen therein; a balloon assembly coupled with the distal end portion of the balloon catheter, the balloon assembly includes: a balloon having distal and proximal balloon ends, the balloon includes inflated and folded deflated configurations, and an elastomer deflation shell coupled along the balloon; and wherein the elastomer deflation shell is configured to stretch in the inflated configuration and to initiate pleating of the balloon with transition from the inflated configuration toward the folded deflated configuration according to the stretching of the elastomer deflation shell.

Example 37 can include, or can optionally be combined with the subject matter of Examples 1-36 to optionally include wherein elastomer deflation shell is continuously coupled across the balloon between the distal and proximal balloon ends.

Example 38 can include, or can optionally be combined with the subject matter of Examples 1-37 to optionally include wherein the elastomer deflation shell is continuously coupled across one or more of a balloon interior or exterior.

Example 39 can include, or can optionally be combined with the subject matter of Examples 1-38 to optionally include wherein the elastomer deflation shell is coupled between a balloon interior and a balloon exterior.

Example 40 can include, or can optionally be combined with the subject matter of Examples 1-39 to optionally include wherein the elastomer deflation shell includes a plurality of deflations struts extending between the distal and proximal balloon ends.

Example 41 can include, or can optionally be combined with the subject matter of Examples 1-40 to optionally include wherein the balloon and the elastomer deflation shell are a coextruded laminate.

Example 42 can include, or can optionally be combined with the subject matter of Examples 1-41 to optionally include wherein the balloon is non-compliant and the elastomer deflation shell is compliant.

Example 43 can include, or can optionally be combined with the subject matter of Examples 1-42 to optionally include wherein the elastomer deflation shell is configured to provide a compressive force around the balloon according to the stretching of the elastomer deflation shell.

Example 44 can include, or can optionally be combined with the subject matter of Examples 1-43 to optionally include wherein the elastomer deflation shell includes stretched and relaxed configurations: in the inflated configuration of the balloon the elastomer deflation shell is in the stretched configuration, and in the folded deflated configuration the elastomer deflation shell is in the relaxed configuration.

Example 45 can include, or can optionally be combined with the subject matter of Examples 1-44 to optionally include wherein the elastomer deflation shell is configured to transition from the stretched configuration to the relaxed configuration at initiating of pleating in the balloon.

Example 46 can include, or can optionally be combined with the subject matter of Examples 1-45 to optionally include wherein the elastomer deflation shell is configured to guide deflation of the balloon toward the folded deflated configuration with initiating of pleating of the balloon. Example 47 can include, or can optionally be combined with the subject matter of Examples 1-46 to optionally include wherein the elastomer deflation shell is configured to decrease bias of the balloon at the initiation of pleating in the balloon.

Example 48 can include, or can optionally be combined with the subject matter of Examples 1-47 to optionally include a balloon system comprising: a balloon catheter extending from a proximal end portion to a distal end portion, the balloon catheter includes an inflation lumen therein; a balloon assembly coupled with the distal end portion of the balloon catheter, the balloon assembly includes a balloon having distal and proximal balloon ends; and a telescoping shaft slidably received in the balloon catheter, the telescoping shaft is coupled with the distal balloon end and the balloon catheter is coupled with the proximal balloon end, and the telescoping shaft is movable between inflation and pleat initiating configurations: in the inflation configuration the distal balloon end is at a first orientation relative to the proximal balloon end, and in the pleat initiating configuration the distal balloon end is at a second orientation relative to the proximal balloon end different from the first orientation, and the second orientation biases the balloon to pleat; and wherein the telescoping shaft transitions between the inflation and pleat initiating configurations according to one or more of longitudinal or rotational movement of the telescoping shaft relative to the balloon catheter.

Example 49 can include, or can optionally be combined with the subject matter of Examples 1-48 to optionally include wherein the first orientation includes the distal and proximal balloon ends at a first spacing, and the second orientation includes the distal and proximal balloon ends at a second spacing greater than the first spacing according to longitudinal movement of the telescoping shaft relative to the balloon catheter.

Example 50 can include, or can optionally be combined with the subject matter of Examples 1-49 to optionally include wherein pleats in the second orientation are aligned with the telescoping shaft between the proximal and distal balloon ends.

Example 51 can include, or can optionally be combined with the subject matter of Examples 1-50 to optionally include wherein the first orientation includes the distal and proximal balloon ends at a first angular orientation, and the second orientation includes the distal and proximal balloon ends relatively rotated to a second angular orientation different than the first angular orientation according to rotation of the telescoping shaft relative to the balloon catheter.

Example 52 can include, or can optionally be combined with the subject matter of Examples 1-51 to optionally include wherein pleats in the second orientation extend helically around the telescoping shaft between the proximal and distal balloon ends.

Example 53 can include, or can optionally be combined with the subject matter of Examples 1-52 to optionally include a balloon system comprising: a balloon catheter extending from a proximal end portion to a distal end portion, the balloon catheter includes an inflation lumen therein; a balloon coupled with the distal end portion of the balloon catheter, the balloon includes distal and proximal balloon ends, the balloon includes inflated and deflated configurations; and a delivery catheter, the balloon and the balloon catheter are slidable within a delivery lumen of the delivery catheter, and the delivery catheter includes: a deformable distal mouth including a resting configuration and an expanded configuration, and an expansion mechanism coupled with the deformable distal mouth, the expansion mechanism is configured to transition the deformable distal mouth between the resting and expanded configurations.

Example 54 can include, or can optionally be combined with the subject matter of Examples 1-53 to optionally include wherein the expansion mechanism includes a braid having proximal and distal braid portions, the distal braid portion is anchored to the deformable distal mouth, and the braid transitions the deformable distal mouth between the resting and expanded configurations with one or more of rotation or longitudinal translation of the braid relative to the delivery catheter.

Example 55 can include, or can optionally be combined with the subject matter of Examples 1-54 to optionally include wherein the braid extends from the deformable distal mouth to a proximal end portion of the delivery catheter.

Example 56 can include, or can optionally be combined with the subject matter of Examples 1-55 to optionally include wherein the proximal braid portion is anchored to an expansion catheter within the delivery catheter, and one or more of rotational or longitudinal translation of the expansion catheter and the braid relative to the delivery catheter transitions the deformable distal mouth between the resting and expanded configurations.

Example 57 can include, or can optionally be combined with the subject matter of Examples 1-56 to optionally include wherein the braid includes a braid, coiled wire, coiled filar or the like.

Example 58 can include, or can optionally be combined with the subject matter of Examples 1-57 to optionally include wherein the braid is slidably coupled relative to the delivery catheter between the distal and proximal braid portions.

Example 59 can include, or can optionally be combined with the subject matter of Examples 1-58 to optionally include one or more deflation guides coupled with the balloon, the one or more deflation guides are configured to bias the balloon toward the deflated configuration.

Example 60 can include, or can optionally be combined with the subject matter of Examples 1-59 to optionally include wherein the deflated configuration is a folded deflated configuration.

Example 61 can include, or can optionally be combined with the subject matter of Examples 1-60 to optionally include a method of using a balloon system comprising: deploying a balloon assembly from a distal mouth of a delivery catheter, the balloon assembly includes a balloon in an initial packaged configuration; inflating the balloon to an inflated configuration, the balloon having an inflated profile larger than a mouth profile of the distal mouth; deflating the balloon from the inflated configuration to a folded deflated configuration having a folded profile smaller than the mouth profile, deflating including: withdrawing an inflation fluid from the balloon, and initiating pleating of the balloon with one or more deflation guides coupled with one or more of the balloon or a balloon catheter; and retracting the deflated balloon in the folded deflated configuration with the folded profile through the distal mouth having the mouth profile.

Example 62 can include, or can optionally be combined with the subject matter of Examples 1-61 to optionally include wherein inflating the balloon includes stretching the one or more deflation guides from a relaxed configuration to a stretched configuration.

Example 63 can include, or can optionally be combined with the subject matter of Examples 1-62 to optionally include wherein stretching the one or more deflation guides from the relaxed configuration to the stretched configuration includes stretching the one or more deflation guides proximate to when the balloon assumes the inflated profile.

Example 64 can include, or can optionally be combined with the subject matter of Examples 1-63 to optionally include wherein initiating pleating of the balloon includes transitioning the one or more deflation guides from the stretched configuration to the relaxed configuration.

Example 65 can include, or can optionally be combined with the subject matter of Examples 1-64 to optionally include wherein deflating the balloon to the folded deflated configuration includes after initiating of pleating of the balloon: folding of the balloon according to the initiated pleating, and continued withdrawal of inflation fluid.

Example 66 can include, or can optionally be combined with the subject matter of Examples 1-65 to optionally include wherein initiating pleating of the balloon includes applying a compressive force at one or more locations on the balloon with the one or more deflation guides.

Example 67 can include, or can optionally be combined with the subject matter of Examples 1-66 to optionally include wherein applying the compressive force at one or more locations on the balloon including applying the compressive force continuously around the balloon with the one or more deflation guides.

Example 68 can include, or can optionally be combined with the subject matter of Examples 1-67 to optionally include wherein applying the compressive force at one or more locations on the balloon including applying the compressive force at discrete locations around the balloon with the one or more deflation guides.

Example 69 can include, or can optionally be combined with the subject matter of Examples 1-68 to optionally include wherein initiating pleating of the balloon with one or more deflation guides includes one or more of longitudinally or rotationally moving a telescoping shaft relative to a balloon catheter between inflation and pleated guiding configurations.

Example 70 can include, or can optionally be combined with the subject matter of Examples 1-69 to optionally include expanding the distal mouth of the delivery catheter with an expansion mechanism.

Example 71 can include, or can optionally be combined with the subject matter of Examples 1-70 to optionally include wherein expanding the distal mouth includes enlarging the distal mouth from the mouth profile to an enlarged mouth profile.

Example 72 can include, or can optionally be combined with the subject matter of Examples 1-71 to optionally include wherein expanding the distal mouth includes one or more of rotating or translating a portion of a braid relative to a delivery catheter and another portion of the braid anchored to the delivery catheter near the distal mouth.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the disclosure can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

The claimed invention is:

1. A balloon system comprising:
a balloon catheter extending from a proximal end portion to a distal end portion, the balloon catheter includes an inflation lumen therein; and
a balloon assembly coupled with the distal end portion of the balloon catheter, the balloon assembly includes:
a balloon having distal and proximal balloon ends, the balloon includes inflated and folded deflated configurations, and
one or more deflation guides coupled with the balloon, the one or more deflation guides are configured to bias the balloon toward the folded deflated configuration.

2. The balloon system of claim 1, wherein the one or more deflation guides includes a plurality of deflation guides coupled along the balloon.

3. The balloon system of claim 1, wherein the one or more deflation guides include at least one deflation strut extending between the proximal and distal balloon ends.

4. The balloon system of claim 3, wherein the at least one deflation strut includes a plurality of deflation struts coupled at distributed locations around the balloon.

5. The balloon system of claim 1, wherein the one or more deflation guides include a deflation shell continuously coupled across the balloon.

6. The balloon system of claim 1, wherein the balloon has a first compliance, and the one or more deflation guides have a second compliance greater than the first compliance.

7. The balloon system of claim 1, wherein the balloon is non-compliant and the one or more deflation guides include an elastomer having a greater compliance than the balloon.

8. The balloon system of claim 1, wherein the one or more deflation guides are configured to provide a compressive force at one or more discrete locations around the balloon to bias the balloon toward the folded deflated configuration.

9. The balloon system of claim 1, wherein the one or more deflation guides are configured to initiate a pleated guiding configuration in the balloon that guides folding of the balloon to the folded deflated configuration.

10. The balloon system of claim 9, wherein the one or more deflation guides are configured to initiate the pleated guiding configuration immediately after the balloon begins deflation.

11. The balloon system of claim 10, wherein the one or more deflation guides are configured to arrest bias of the balloon at the pleated guiding configuration of the balloon.

12. The balloon system of claim 1, wherein the balloon catheter and the balloon assembly include the one or more deflation guides.

13. The balloon system of claim 12, wherein the balloon catheter includes an exterior shaft and the one or more deformation guides include a telescoping interior shaft within the exterior shaft,
the exterior shaft is coupled with the proximal balloon end, and
the interior shaft is coupled with the distal balloon end, the interior shaft is slidable within the exterior shaft, and one or more of relative rotation or longitudinal relative movement of the interior shaft relative to the exterior shaft is configured to bias the balloon toward the folded deflated configuration.

14. The balloon system of claim 1 comprising a delivery catheter, the balloon assembly and the balloon catheter are slidable within a delivery lumen of the delivery catheter, and the delivery catheter includes:
a deformable distal mouth including a resting configuration and an expanded configuration, and
an expansion mechanism coupled with the deformable distal mouth, the expansion mechanism is configured to transition the deformable distal mouth between the resting and expanded configurations.

15. The balloon system of claim 14, wherein the expansion mechanism includes a braid having proximal and distal braid portions, the distal braid portion is anchored to the deformable distal mouth, and the braid transitions the deformable distal mouth with one or more of rotation or longitudinal translation of the braid relative to the delivery catheter.

16. A balloon system comprising:
a balloon catheter extending from a proximal end portion to a distal end portion, the balloon catheter includes an inflation lumen therein;
a balloon assembly coupled with the distal end portion of the balloon catheter, the balloon assembly includes:
a balloon having distal and proximal balloon ends, the balloon includes inflated and folded deflated configurations, and
one or more elastomer deflation struts coupled with the balloon between the distal and proximal balloon ends; and
wherein the one or more elastomer deflation struts are configured to stretch in the inflated configuration of the balloon and to initiate pleating of the balloon with transition from the inflated configuration toward the folded deflated configuration according to the stretching of the one or more elastomer deflation struts.

17. The balloon system of claim 16, wherein the one or more elastomer deflation struts are within the balloon.

18. The balloon system of claim 16, wherein the one or more elastomer deflation struts are coupled along the balloon.

19. The balloon system of claim 18, wherein at least a portion of each of the one or more elastomer deflation struts are slidably coupled along the balloon.

20. The balloon system of claim 18, wherein at least a portion of each of the one or more elastomer deflation struts is anchored along the balloon.

21. The balloon system of claim 16, wherein the balloon has a first compliance and the one or more elastomer deflation struts have a second compliance greater than the first compliance.

22. The balloon system of claim 16, wherein the one or more elastomer deflation struts are configured to provide a compressive force at one or more discrete locations around the balloon according to the stretching of the one or more elastomer deflation struts.

23. The balloon system of claim 16, wherein the one or more elastomer deflation struts include stretched and relaxed configurations:
in the inflated configuration of the balloon the one or more elastomer deflation struts are in the stretched configuration, and
in the folded deflated configuration the one or more elastomer deflation struts are in the relaxed configuration.

24. The balloon system of claim 23, wherein the one or more elastomer deflation struts are configured to transition from the stretched configuration to the relaxed configuration at initiating of pleating.

25. The balloon system of claim 16, wherein the one or more elastomer deflation struts are configured to guide deflation of the balloon toward the folded deflated configuration with initiating of pleating of the balloon.

26. A method of using a balloon system comprising:
deploying a balloon assembly from a distal mouth of a delivery catheter, the balloon assembly includes a balloon in an initial packaged configuration;
inflating the balloon to an inflated configuration, the balloon having an inflated profile larger than a mouth profile of the distal mouth;
deflating the balloon from the inflated configuration to a folded deflated configuration having a folded profile smaller than the mouth profile, deflating including:
withdrawing an inflation fluid from the balloon, and
initiating pleating of the balloon with one or more deflation guides coupled with one or more of the balloon or a balloon catheter; and
retracting the deflated balloon in the folded deflated configuration with the folded profile through the distal mouth having the mouth profile.

27. The method of claim 26, wherein inflating the balloon includes stretching the one or more deflation guides from a relaxed configuration to a stretched configuration.

28. The method of claim 27, wherein stretching the one or more deflation guides from the relaxed configuration to the stretched configuration includes stretching the one or more deflation guides proximate to when the balloon assumes the inflated profile.

29. The method of claim 27, wherein initiating pleating of the balloon includes transitioning the one or more deflation guides from the stretched configuration to the relaxed configuration.

30. The method of claim 26, wherein deflating the balloon to the folded deflated configuration includes after initiating of pleating of the balloon:
folding of the balloon according to the initiated pleating, and
continued withdrawal of inflation fluid.

31. The method of claim 26, wherein initiating pleating of the balloon includes applying a compressive force at one or more locations on the balloon with the one or more deflation guides.
